(19) United States
(12) Patent Application Publication
(10) Pub. No.: US 2022/0233458 A1
(43) Pub. Date: Jul. 28, 2022

(54) Complex of Noble Metal Oxide Nanoparticles and Tellurium Nanowires and Biomedical Applications Thereof

(71) Applicant: Northeastern University, Boston, MS (US)

(72) Inventors: David Medina CRUZ, Jamaica Plain, MA (US); Ada Vernet CRUA, Brighton, MA (US); Thomas J. WEBSTER, Barrington, RI (US)

(21) Appl. No.: 17/608,538

(22) PCT Filed: Jun. 1, 2020
(86) PCT No.: PCT/US20/35609

§ 371 Date: Nov. 3, 2021

Related U.S. Application Data
(60) Provisional application No. 62/855,798, filed on May 31, 2019.

(51) Int. Cl. A61K 9/51; A61K 47/69; A61K 33/04; A61P 31/04

(52) U.S. Cl. CPC A61K 9/5115; A61K 47/6929; A61P 31/04; A61K 33/04; A61K 9/5192; A61K 47/6953

(57) ABSTRACT

Green-synthesized tellurium nanowires (GREEN-TeNWs) are generated using a biopolymer as a unique reducing agent, purified, and used as a template for the growth of coated palladium nanoparticles (PdNPs) and platinum nanoparticles (PtNPs) on top of the GREEN-TeNWs, in a reaction that can take place in seconds, with no need for high temperature, stirring, or for additional reducing agent. The heterogeneous structure can contain palladium oxide or platinum oxide. The green-synthesized PdNPs-TeNWs (palladium nanoparticles with tellurium nanowires) and PtNPs-TeNWs (platinum nanoparticles with tellurium nanowires) show potential biomedical applications as antibacterial, anticancer, and antioxidant agents, and show low cytotoxicity for healthy human cells.

### CROSS-REFERENCE TO RELATED APPLICATIONS

This application claims priority to U.S. Provisional Application No. 62/855,798, filed 31 May 2019, the entirety of which is incorporated herein by reference.

### BACKGROUND

Antimicrobial resistance to antibiotics (AMR) and cancer are two major concerns that the healthcare system is currently trying to address, with a huge impact in society. Both AMR and cancer are significantly increasing. The extent of the incidence of both diseases in society not only affects the healthcare system, but also the economy. When AMR is considered, it is clear that the current use of antibiotics is leading to an exponential increase of new reports of bacteria becoming resistant to them. Misuse, overuse, and self-treatments are extremely related to the increase in incidence of bacterial infections that were easily treated in the past but now can easily kill again.

Current cancer treatments are based on chemotherapy, radiotherapy, and the use of both for stopping the growth and spreading of the tumors. However, these methods are not free of drawbacks, such as severe side-effects and indirect targeting and damage to healthy tissue surrounding the cancerous cells, sometimes leading to premature death of the patients. Beyond current treatments, new approaches, such as immunotherapy or gene therapy are presented as a potential solution to the associated problem. Nevertheless, most of the related processes are still in development, while they are also too expensive and not affordable for most patients. Besides, similar to the resistance behavior shown by bacterial populations after continuous treatment with antibiotics, cancer cells and tumors have been reported to become resistant to chemotherapy drugs after exposure (Housman et al., Cancers, 2014). During treatment, some of the cells that are treated with the drugs are not killed, experiencing mutations. The mechanisms that lead to this behavior are not extensively understood, yet they are related to many factors, such as individuals' genetic differences. Multi-drug resistance, cell death inhibition by apoptosis suppression, alteration of the drug metabolism or enhancement of the DNA repair and gene amplification processes are some of the mechanisms that have been reported in literature.

Therefore, new approaches far beyond the current use of antibiotics, chemotherapy, and radiotherapy treatments are needed, and the exponential rise of the nanotechnological view of medicine may offer a potential solution. Nanostructures, with a high-surface-to-volume ratio, a highly reactive surface and tunability, may present a solution to overcome uptake, resistance, and bioavailability issues presented in current treatments. Some formulations have been known as powerful antibacterial and anticancer agents, such as silver (Ag) and gold (Au) nanoparticles. Despite some efficiency as biomedical agents, the use of nanomaterials is related to drawbacks, many in terms of synthesis, because how the structures are synthesized is related to their applications and impact in the environment. Production of nanomaterials has been accomplished using physicochemical methodologies, such as chemical vapor deposition (CVD), redox reactions, and laser ablation. Despite the efficiency of these methods, they are often associated with highly toxic by-products, extreme reaction conditions, use of harsh and contaminating chemicals and biocompatibility problems once they are released in biological tissue.

There is a need for new technologies that provide both a holistic approach to synthesis of nanoparticles and to the directed treatments to clarify the role of the physico-chemical properties of nanoparticles in determining their toxicological behavior. Moreover, it is important to carry out investigations focused on environmental and biological monitoring to verify and validate experimental biomarkers of exposure and early effect in real exposure contexts, elucidating the role of these nanomaterials within biological interactions.

### SUMMARY

Green-synthesized tellurium nanowires (GREEN-TeNWs) are generated using starch as a reducing agent for tellurium, purified, and used as a template for the growth of palladium nanoparticles (PdNPs) and platinum nanoparticles (PtNPs) on top of the GREEN-TeNWs, in a reaction that can take place in seconds, with no need for high temperature, stirring, and for additional reducing agent. The heterogeneous structure is extensively characterized in terms of morphology, compositions, and surface chemistry using TEM, SEM, EDX, XPS, XRD and FTIR techniques. The green-synthesized PdNPs-TeNWs and PtNPs-TeNWs are tested for their potential biomedical applications as antibacterial, anticancer, and antioxidant agents, and show low cytotoxicity for healthy human cells. The synergetic structures show enhanced biomedical applications, demonstrating that the combination of the metal nanoparticles and the metalloid nanowires can be used as therapeutic agents.

The present technology can be further summarized by the following features.

1. A method of inhibiting proliferation of pathogenic cells in a subject, the method comprising administering to the subject coated metal nanoparticles attached to tellurium nanowires, wherein the tellurium nanowires have a wire core comprising tellurium, and wherein the coated metallic nanoparticles have a metallic core and an outer coating comprising a polymer, whereby proliferation of the pathogenic cells is inhibited.
2. The method of feature 1, wherein the pathogenic cells are cancer cells and proliferation of the cancer cells is inhibited at least twice as much as proliferation of non-cancerous cells is inhibited in the subject.
3. The method of feature 1, wherein the pathogenic cells are bacterial cells or drug resistant bacterial cells.
4. The method of any of the preceding features, wherein the polymer is a biopolymer isolated from a naturally occurring biological material.
5. The method of feature 4, wherein the biopolymer is starch.
6. The method of feature 1, wherein the polymer is a synthetic polymer.
7. The method of feature 6, wherein the synthetic polymer is polyethylene glycol.
8. The method of any of the preceding features, wherein the wire core comprises tellurium hexagonal crystal structure.
9. The method of any of the preceding features, wherein at least a portion of the tellurium nanowires have a star-shaped structure comprising tellurium nanowires radiating outward from a central point.
10. The method of any of the preceding features, wherein the tellurium nanowires have a diameter of about 15 nm to about 35 nm.
11. The method of any of the preceding features, wherein the coated metal nanoparticles each have a size in the range from about 35 nm to about 120 nm.
12. The method of any of the preceding features, wherein the coated metallic nanoparticles have a coating that is about 1 nm thick.
13. The method of any of the preceding features, wherein the metallic core comprises a metal oxide.
14. A method of producing polymer-coated metal nanoparticles attached to tellurium nanowires, the method comprising:

(a) mixing telluric acid (H₂TeO₄) with an aqueous polymer solution or suspension to obtain a mixture of telluric acid, polymer, and water;

(b) heating the mixture in a sealed vessel at a temperature from about 120° C. to about 200° C. for about 2 hours to about 20 hours; whereby tellurium nanowires coated with the polymer are produced;

(c) centrifuging the product from step (b) to obtain a pellet;

(d) resuspending the pellet in water; and

(e) adding a metallic salt to the suspension and waiting for a reaction time, whereby polymer-coated metal nanoparticles attached to tellurium nanowires are produced.

15. The method of feature 14, further comprising:

(f) centrifuging the product from step (e) to obtain a pellet;

(g) resuspending the pellet in water; and

(h) lyophilizing the resuspended pellet.

16. The method of feature 14 or 15, wherein the reaction time is about 1 minute.
17. The method of any of features 14-16, wherein the polymer-coated metal nanoparticles comprise a metal oxide.
18. The method of any of features 14-17, wherein the metallic salt is palladium chloride (PdCl₂), potassium tetrachloroplatinate (K₂PtCl₄), or combinations thereof.
19. The method of any of features 14-18, wherein the polymer is starch.
20. The method of any of features 14-19, wherein the temperature in step (b) is about 160° C.
21. The method of any of features 14-20, wherein the heating in step (b) is carried out for about 15 hours.
22. The method of any of features 14-21, wherein the tellurium nanowires coated with the polymer are produced in the form of star-shaped nanostructures, each star-shaped nanostructure comprising a central cluster and a plurality of coated tellurium nanowires extending from the central cluster.
23. The method of any of features 14-22, wherein the resulting tellurium nanowires each have a diameter of about 15 nm to about 35 nm.
24. The method of any of features 14-23, wherein the coated metal nanoparticles each have a size in the range from about 35 nm to about 120 nm.
25. The method of any of features 14-24, further comprising;

(e1) sonicating the polymer-coated metal nanoparticles attached to tellurium nanowires to release the polymer-coated metal nanoparticles from the tellurium nanowires.

26. The method of feature 19, wherein the produced tellurium nanowires comprise a core comprising tellurium in a hexagonal tellurium crystal form and the metallic nanoparticles have a coating comprising starch.
27. Tellurium nanowires having a core comprising tellurium and metallic nanoparticles with an outer coating comprising a polymer made by a method of any one of features 14-24.
28. The tellurium nanowires of feature 27, wherein the tellurium nanowires do not comprise amorphous tellurium.

As used herein, minimum inhibitory concentration (MIC) is the lowest concentration of a nanoparticle that will inhibit, in vitro, the visible (or measurable) growth of a cell or microorganism after 24 hours of incubation. The half maximal inhibitory concentration (IC₅₀) is the concentration of a nanoparticle that is needed to inhibit, in vitro, the growth of a cell or microorganism by 50%. The chemical “MTS” utilized in MTS assays described herein refers to MTS (3-(4,5-dimethylthiazol-2-yl)-5-(3-carboxymethoxyphenyl)-2-(4-sulfophenyl)-2H-tetrazolium).

As used herein, metal nanoparticles refers to nanoparticles comprising metals, metalloids (e.g., silicon), metal oxides, and combinations thereof. As used herein, a nanoparticle is a particle that has at least one dimension on the nanoscale. For example, a GREEN-TeNW (green-synthesized tellurium nanowire) with metal nanoparticles can have a width of nanometers and a length of millimeters.

As used herein, an pathogenic cell is any cell that is not a normal, healthy cell within an organism. Examples of pathogenic cells are invasive bacterial cells, fungal cells, normal cells infected with a virus, and cancer cells.

As used herein, the term “about” and “approximately” are defined to be within 10%, 5%, 1%, or 0.5% of the stated value. As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expressions “consisting essentially of” or “consisting of.”.

### BRIEF DESCRIPTION OF THE DRAWINGS

FIG. 1A shows a transmission electron microscopy (TEM) image of PdNPs-TeNWs (palladium nanoparticles-tellurium nanowires) formed with green synthesized tellurium nanowires (GREEN-TeNWs).

FIG. 1B shows a TEM image of PtNPs (platinum nanoparticles-tellurium nanowires) formed with green synthesized tellurium nanowires (GREEN-TeNWs).

FIG. 1C shows a scanning electron microscopy (SEM, 10 kX) image of green synthesized tellurium nanowires (GREEN-TeNWs) without nanoparticles on the nanowires.

FIG. 1D shows an SEM image of GREEN-TeNWs without nanoparticles on the nanowires at higher magnification (25 kX) than FIG. 1C.

FIG. 2A shows an SEM image of PdNPs-TeNWs formed with GREEN-TeNWs.

FIG. 2B shows an SEM image of PtNPs-TeNWs formed with green synthesized tellurium nanowires (GREEN-TeNWs).

FIG. 3A shows an SEM image of PtNPs-TeNWs formed with green synthesized tellurium nanowires (GREEN-TeNWs) with positions or spots indicated for targeted EDX (energy-dispersive X-ray spectroscopy) analysis.

FIG. 3B shows a high-resolution SEM image of the PtNPs-TeNWs formed with GREEN-TeNWs shown in FIG. 3A.

FIG. 4A shows in the top spectrum the EDX (energy-dispersive X-ray spectroscopy) analysis of position 2 (EDS Spot 2) indicated in FIG. 3A. The center spectrum is the EDX analysis of position 1 (EDS Spot 1) indicated in FIG. 3A. The bottom spectrum is the EDX analysis of the full area position (Full Area 1) indicated in FIG. 3A.

FIG. 4B shows the atomic content (%) from the EDX analysis of position 2 (EDS Spot 2) indicated in FIG. 3A. Elements detected are shown at left on the Y-axis.

FIG. 4C shows the atomic content (%) from the EDX analysis of position 1 (EDS Spot 1) indicated in FIG. 3A. Elements detected are shown at left on the Y-axis.

FIG. 4D shows the atomic content (%) from the EDX analysis of the full area position (Full Area 1) indicated in FIG. 3A. Elements detected are shown at left on the Y-axis.

FIG. 5A shows an SEM image of PdNPs formed with GREEN-TeNWs with positions or spots selected for EDX analysis: EDS Spot 1 at left, Full Area 1 in center, and EDS Spot 2 at top.

FIG. 5B shows a high-resolution SEM image of the PdNPs formed with GREEN-TeNWs shown in FIG. 5A.

FIG. 6A shows in the top spectrum the EDX analysis of position 2 (EDS Spot 2) indicated in FIG. 5A. The center spectrum is the EDX analysis of position 1 (EDS Spot 1) indicated in FIG. 5A. The bottom spectrum is the EDX analysis of the full area position (Full Area 1) indicated in FIG. 5A.

FIG. 6B shows the atomic content (%) from the EDX analysis of position 2 (EDS Spot 2) indicated in FIG. 5A. Elements detected are shown at left on the Y-axis.

FIG. 6C shows the atomic content (%) from the EDX analysis of position 1 (EDS Spot 1) indicated in FIG. 5A. Elements detected are shown at left on the Y-axis.

FIG. 6D shows the atomic content (%) from the EDX analysis of the full area position (Full Area 1) indicated in FIG. 5A. Elements detected are shown at left on the Y-axis.

FIG. 7 shows an overlayed comparison of the FT-IR spectra of tellurium nanowires with platinum nanoparticles (TeNWs-PtNPs, a, top spectrum), TeNWs-PdNPs (b, center spectrum, tellurium nanowires with palladium nanoparticles), and potato starch (c, bottom spectrum). The IR spectra were acquired in ATR (attenuated total reflectance) mode.

FIG. 8 shows a comparison between the experimental X-ray powder diffraction (XRD) patterns for TeNWs-PtNPs (top spectrum) and TeNWs-PdNPs (second from top spectrum). The calculated XRD pattern for cubic PdO (NaCl-type) is shown in the second from bottom spectrum, and the calculated XRD pattern for cubic NaCl is shown in the bottom spectrum.

FIG. 9 shows X-ray photoelectron spectroscopy (XPS) spectra of PdNPs-TeNWs (palladium nanoparticles synthesized with green tellurium nanowires, top spectrum) and PtNPs-TeNWs (platinum nanoparticles synthesized with green tellurium nanowires, bottom spectrum). The wide scans have been normalized arbitrarily to the C 1s core level peak for comparison. Main features have been identified in the spectra.

FIG. 10A shows XPS core level spectra at the Te 3d region for PtNPs-TeNWs (top trace) and PdNPs-TeNWs (bottom trace).

FIG. 10B shows XPS core level spectra at the Pd 3d region for PdNPs-TeNWs.

FIG. 10C shows XPS core level spectra at the Pt 4f region for PtNPs-TeNWs.

FIG. 11A shows XPS core level spectra at the C 1s region for PtNPs-TeNWs (top spectrum) and for PdNPs-TeNWs (bottom spectrum).

FIG. 11B shows XPS core level spectra at the O 1s region for PtNPs-TeNWs (top spectrum) and for PdNPs-TeNWs (bottom spectrum).

FIG. 12A shows a TEM image of PdNPs (palladium nanoparticles) formed with green synthesized tellurium nanowires (GREEN-TeNWs) taken 60 days after synthesis.

FIG. 12B shows a TEM image of PtNPs (platinum nanoparticles) formed with green synthesized tellurium nanowires (GREEN-TeNWs) taken 60 days after synthesis.

FIG. 13A shows cultured bacterial proliferation of multi-drug resistant (MDR) E. coli treated with concentrations in the range from 0 to 100 μg/mL PdNPs-TeNWs for 28 hours.

FIG. 13B shows cultured bacterial proliferation of MDR E. coli treated with concentrations in the range from 0 to 100 μg/mL PtNPs-TeNWs for 28 hours.

FIG. 13C shows cultured bacterial proliferation of methicillin-resistant Staphylococcus aureus (MRSA) treated with concentrations in the range from 0 to 100 μg/mL PdNPs-TeNWs for 28 hours.

FIG. 13D shows cultured bacterial proliferation of MRSA treated with concentrations in the range from 0 to 100 μg/mL PtNPs-TeNWs for 28 hours.

FIG. 14A shows a plot of parameter A (from the modified Gompertz Equation 2) versus nanoparticle concentration (μg/mL) for PdNPs-TeNWs (▪) and PtNPs-TeNWs (●) with MDR E. coli. Parameter A represents maximum bacterial growth.

FIG. 14B shows a plot of parameter A (from the modified Gompertz Equation 2) versus nanoparticle concentration (μg/mL) for PdNPs-TeNWs (▪) and PtNPs-TeNWs (●) with MRSA (methicillin-resistant Staphylococcus aureus).

FIG. 14C shows a plot of parameter p (from the modified Gompertz Equation 2) versus nanoparticle concentration (μg/mL) for PdNPs-TeNWs (▪) and PtNPs-TeNWs (●) with MDR E. coli. Parameter p represents maximum bacterial growth rate.

FIG. 14D shows a plot of parameter p (from the modified Gompertz Equation 2) versus nanoparticle concentration (μg/mL) for PdNPs-TeNWs (▪) and PtNPs-TeNWs (●) with MRSA.

FIG. 14E shows a plot of parameter k (from the modified Gompertz Equation 2) versus nanoparticle concentration (μg/mL) for PdNPs-TeNWs (▪) and PtNPs-TeNWs (●) with MDR E. coli. Parameter k represents lag time in the bacterial growth, or the duration of time where bacteria are adapting themselves to the growth conditions offered by the media.

FIG. 14F shows a plot of parameter k (from the modified Gompertz Equation 2) versus nanoparticle concentration (μg/mL) for PdNPs-TeNWs (▪) and PtNPs-TeNWs (●) with MRSA.

FIG. 15A shows a plot of colony counting unit assay conducted over MDR E. coli with concentrations from 0 to 100 μg/mL PdNPs-TeNWs.

FIG. 15B shows a plot of colony counting unit assay conducted over MDR E. coli with concentrations from 0 to 100 μg/mL PtNPs-TeNWs.

FIG. 15C shows a plot of colony counting unit assay conducted over MRSA with concentrations from 0 to 100 μg/mL PdNPs-TeNWs.

FIG. 15D shows a plot of colony counting unit assay conducted over MRSA with concentrations from 0 to 100 μg/mL PtNPs-TeNWs.

FIG. 16A shows plots of cell proliferation (cells/mL) versus concentration PdNPs-TeNWs (μg/mL) for human dermal fibroblast (HDF) cells. The left bar plotted for each concentration is Day 1; the center bar plotted for each concentration is Day 3; the right bar plotted for each concentration is Day 5.

FIG. 16B shows plots of cell proliferation (cells/mL) versus concentration PtNPs-TeNWs (μg/mL) for human dermal fibroblast (HDF) cells. The left bar plotted for each concentration is Day 1; the center bar plotted for each concentration is Day 3; the right bar plotted for each concentration is Day 5.

FIG. 17A shows plots of cell proliferation (cells/mL) versus concentration PdNPs-TeNWs (μg/mL) for human melanoma cells. The left bar plot for each concentration is Day 1; the center bar plot for each concentration is Day 3; the right bar plot for each concentration is Day 5.

FIG. 17B shows plots of cell proliferation (cells/mL) versus concentration PtNPs-TeNWs (μg/mL) for human melanoma cells. The left bar plot for each concentration is Day 1; the center bar plot for each concentration is Day 3; the right bar plot for each concentration is Day 5.

FIG. 18A shows a scanning electron microscopy (SEM) image of control (untreated) MDR E. coli cells.

FIG. 18B shows an SEM image of MDR E. coli cells after treatment with PdNPs-TeNWs.

FIG. 18C shows an SEM image of MDR E. coli cells after treatment with PtNPs-TeNWs.

FIG. 18D shows an SEM image of control (untreated) MRSA (methicillin-resistant Staphylococcus aureus) cells.

FIG. 18E shows an SEM image of MRSA cells after treatment with PdNPs-TeNWs.

FIG. 18F shows an SEM image of MRSA cells after treatment with PtNPs-TeNWs.

FIG. 19A shows an SEM image of control (untreated) HDF cells.

FIG. 19B shows an SEM image of HDF cells after treatment with PdNPs-TeNWs.

FIG. 19C shows an SEM image of HDF cells after treatment with PtNPs-TeNWs.

FIG. 19D shows an SEM image of control (untreated) human melanoma cells.

FIG. 19E shows an SEM image of human melanoma cells after treatment with PdNPs-TeNWs.

FIG. 19F shows an SEM image of human melanoma cells after treatment with PtNPs-TeNWs.

FIG. 20A shows a plot of reactive oxygen species (ROS) measured by fluorescence intensity (Y-axis) versus PdNPs-TeNWs concentration (μg/mL, X-axis) for human melanoma cells after 24 hours.

FIG. 20B shows a plot of reactive oxygen species (ROS) measured by fluorescence intensity (Y-axis) versus PtNPs-TeNWs concentration (μg/mL, X-axis) for human melanoma cells after 24 hours.

### DETAILED DESCRIPTION

Green tellurium nanowire (Green-TeNW) synthesis of PdNPs (palladium nanoparticles) and PtNPs (platinum nanoparticles) is accomplished without addition of reducing and capping agents to reduce the metal ions to elemental valence state, to bond nanoparticles to the wire nanostructure, and to avoid aggregation of the nanoparticles. For synthesis of the nanoparticles, the green-synthesis reaction can take place in about a minute, with no need for high temperature and stirring.

For a green synthetic route to synthesize tellurium nanowires, telluric acid (H₂TeO₄) can be mixed and stirred with a starch solution in deionized water. The resulting mixture can be heated in a sealed reaction vessel at a reaction temperature from about 120° C. to about 200° C., or about 140° C. to about 180° C. for a reaction time period of about 2 to about 20 hours, about 6 to about 18 hours, or about 10 to about 18 hours. Isolation and purification can be done by any means known in the art, and centrifugation with water rinsing is a non-limiting example. The starch is believed to reduce the metal, resulting in formation of the tellurium nanowires. For a green synthesis route, a suitable amount of telluric acid (H₂TeO₄) can be mixed with about 0.15 g of a starch solution in deionized water. Then, the mixture can be transferred into a Teflon-lined stainless-steel reactor and placed into an oven at about 160° C. for about 15 hours. After the reaction, the mixture can be allowed to cool down at room temperature. A non-limiting example of a green synthetic route is shown in Reaction 1.

H₂TeO₄+(C₆H₁₀O₅)ₙ(H₂O) 15h/160° C.>GREEN-TeNWs (1)

Reaction 1. Example reaction for the synthesis of TeNWs by green synthesis (1).

After the mixture is transferred to a teflon-lined stainless steel reactor and placed into an oven at about 160° C. for about 15 hours, the mixture is then allowed to cool down to room temperature. The mixture can then be centrifuged, washed, and lyophilized. For example, the nanowire solution can be centrifuged at about 10,000 rpm for about 20 minutes, with the pellet subsequently washed twice with water and centrifuged again at the same rate and time. The precipitate can be re-suspended in deionized water, frozen at −80° C. for 4 hours, and lyophilized overnight. The final powder of GREEN-TeNWs can be re-suspended in a suitable amount of deionized, sterile water to reach the final concentration needed for further experiments.

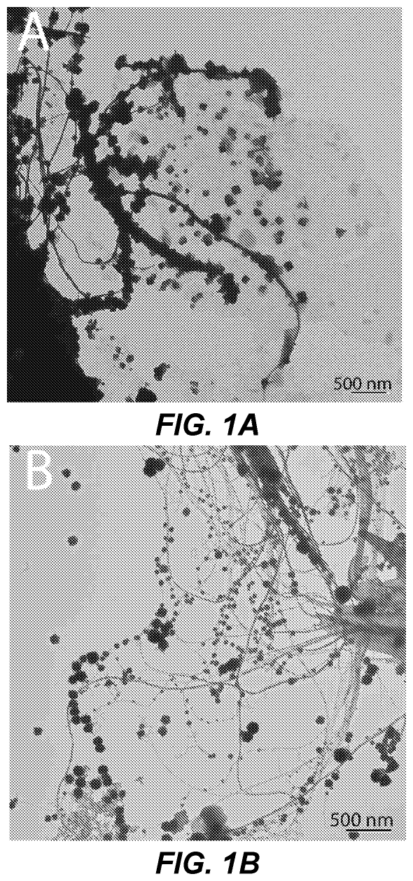

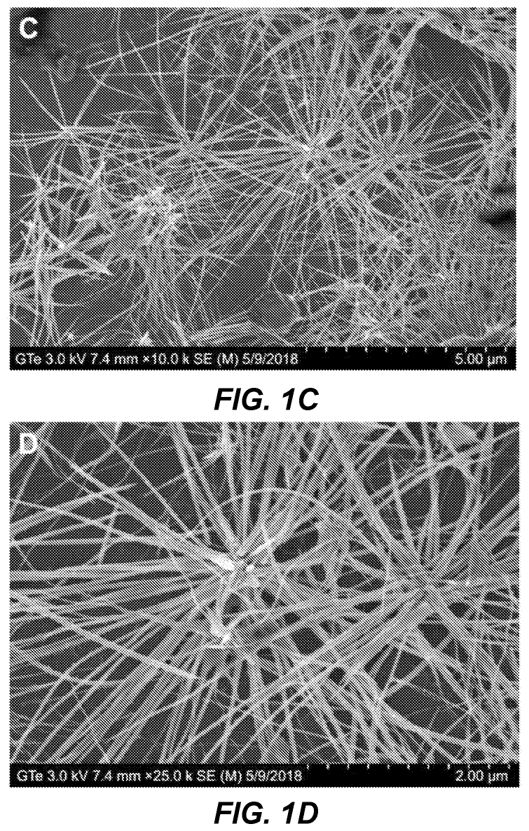

As can be seen in FIGS. 1C-1D, at least some of the GREEN-TeNWs can show growth initiated from a point with extended growth radiating from the central point. The GREEN-TeNWs can grow from a cluster and extend for several micrometers, with a star-shaped uniform structure. The star-shaped uniform nanostructures can arise due to the green synthesis, and the GREEN-TeNWs also have a unique polymer coating, which is believed to subsequently cause the fast synthesis of the palladium and platinum nanoparticles.

The polymer used to coat the tellurium nanowires can be a biopolymer isolated from a naturally occurring biological material, and optionally modified, such as by cross-linking, heat treatment, or chemical modification. For example, the polymer can be starch, or cross-linked starch, or starch modified by heat treatment, such as gelatinized starch. The polymer also can be a synthetic polymer, which is preferably non-toxic and biodegradable, such as polyethylene glycol. The core of the nanowires contains or consists essentially of tellurium hexagonal crystal structure; preferably, the core does not contain amorphous tellurium. The coated tellurium nanowires can be present as individual, non-associated nanowires, or at least a portion of the nanowires can have a star-shaped structure comprising coated tellurium nanowires radiating outwards from a central point, where the nanowires are clustered together. The coated tellurium nanowires (GREEN-TeNWs) can have a diameter of about 1 nm to about 40 nm, about 25±8 nm, or about 15 to about 35 nm, and each GREEN-TeNW can extend for several microns. Of course, depending on the severity of the conditions used for isolation, purification, or lyophilization, the GREEN-TeNWs could be broken into smaller lengths. The polymer coating, such as a starch coating, can be at least 1 nm thick.

Tellurium has two allotropes, crystalline (hexagonal) and amorphous. It is known in the art that amorphous tellurium has a brown-black color and a powder morphology, and hexagonal crystalline tellurium has a silvery lustrous gray color with rigid crystals, and can have chains of Te atoms, that can form durable nanostructures of varying lengths. XRD studies of the GREEN-TeNWs (not shown) support hexagonal crystalline form and the presence of foreign phases related to Te-based oxides compounds. Amorphous Te is not detected in the XRD of GREEN-TeNWs, but generally the limit of detection for amorphous forms in XRD can be challenging because amorphous materials form an amorphous “hump” in the XRD spectrum. The hump can be difficult to detect under the sharp, intense crystalline peaks. Amorphous Te would form nanoparticles and not nanowires extending for microns with a width of about 1 nm to about 40 nm, 25±8 nm (or about 15 to about 35 nm). The GREEN-TeNWs have a coating that includes starch from the green synthesis. Depending on the analytical technique and the polymer used, the coating can be shown as poly(ethylene glycol) or [(CH₂)₂O]n, and direct comparison by FT-IR (in ATR mode) shows characteristic IR bands, which will be discussed in more detail (with the Pd/Pt nanoparticles) below. The GREEN-TeNWs can be starch-functionalized with a starch coating. The thickness of the coating can be about at least 1 nm thick, but the thickness can depend on reaction conditions of example Reaction 1 shown above. For example, if larger amounts of excess polymer are added to the reaction, thicker residual coatings of starch are expected. Also, the thickness of the coating could be changed by the isolation and purification conditions for the GREEN-TeNWs. Less starch-coating can be found if the starch is all used in the synthetic process.

The GREEN-TeNWs can be used as quick reducing agents for the production of coated nanoparticles. In the following example for the reduction of Pt and Pd ions to their elemental form, the corresponding metallic salt can be added into a mixture of previously synthesized GREEN-TeNWs and water. A solution of about 1:1:15 in volume of newly synthesized GREEN-TeNWs, 5 mg/mL of metallic salt precursor, and distilled water can be prepared. Non-limiting examples of metallic salt precursors that can be used are palladium chloride (PdCl2) and potassium tetrachloroplatinate (K₂PtCl₄). The mixture can then be allowed to react at about room temperature for about 1 minute, with no heat or agitation. The reaction can then be transferred, centrifuged, and washed with distilled water at about 10000 rpm for about 20 min. The re-suspended solution can be lyophilized, resulting in a black powder, that can be used in suspension form or otherwise, for further characterization and experiments. The thickness of the coating on the coated nanoparticles can be about 1 nm, but the thickness can depend on, for example, reaction conditions, excess starch or polymer, reduction, and time. For example, if larger amounts of excess polymer are added to the reaction, thicker coatings of polymer are expected. The thickness of the coating could be changed by the isolation and purification conditions, and sonication, discussed below, depending on power applied could affect the thickness. Less starch-coating can be found if the starch is all used in the synthetic process.

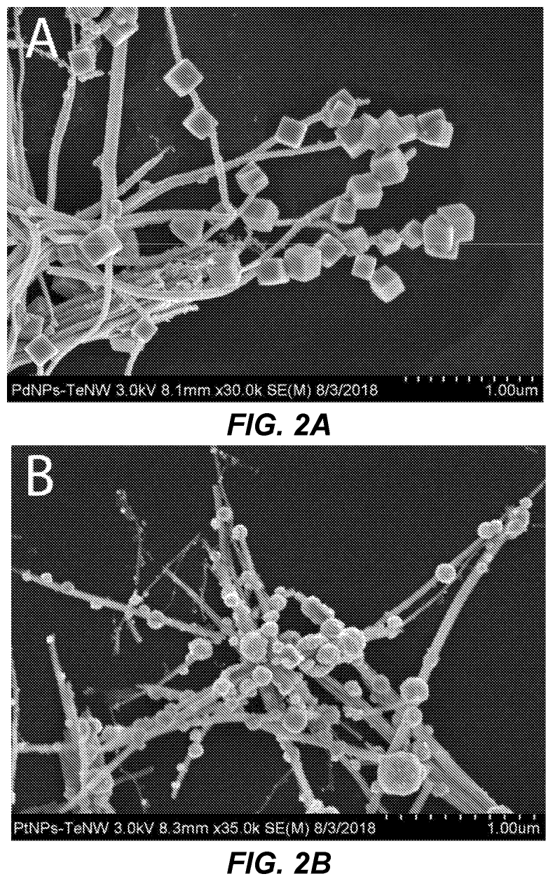

Transmission electron microscope (TEM) and SEM images of PdNPs-TeNWs, taken right after purification of samples, are shown in FIG. 1A (TEM) and FIG. 2A (SEM). TEM/SEM images of PtNPs-TeNWs are shown in FIG. 1B (TEM) and in FIG. 2B (SEM). Different morphologies and features are found between palladium nanoparticles (PdNPs) and platinum nanoparticles (PtNPs) when the nanomaterials are characterized by TEM and SEM. As can be seen in the figures, the TeNWs serve as a template for the growth of PdNPs and PtNPs, and the nanoparticles grow on the surface of the nanowires. The nanoparticle structures grow all over the length of the nanowires, developing a narrow size distribution and a well-defined shape. The SEM characterization of PdNP-TeNWs (FIG. 2A) shows a homogeneous cubic-shaped structure while for the PtNP-TeNWs (FIG. 2B) spherical-shaped structures are clearly observed along the surface of the elongated Te nanowires. In an analysis of FIGS. 1A/2A, the PdNPs show a square shape and an average size of about 80±26 nm. In an analysis of FIGS. 1B/2B, the PtNPs show a spherical shape and an average size of about 68±29 nm. The coated metal nanoparticles can have a size in the range from about 1 nm to about 150 nm, from about 35 nm to about 120 nm, from about 39 nm to about 106 nm, or about 40 nm to about 110 nm, from about 39 nm to about 97 nm, or about 40 nm to about 100 nm, from about 54 nm to about 106 nm, or about 50 nm to about 110 nm; depending on how long the reaction proceeds and reaction conditions. When the particles reach enough size, some of them are released while others remain attached to the nanowire structure. Further characterization was conducted to observe the strength of the bonding between the nanoparticles and nanowires. Upon application of sonication, a release of the nanoparticles was observed. The released nanoparticles remained monodispersed in solution with a low degree of aggregation, remaining stable. The coated metal nanoparticles can be formed on the GREEN-TeNWs in a reaction time under about a minute, for example in about 5 seconds to about 60 seconds, in about 15 seconds to about 60 seconds, or in about 30 seconds to about 60 seconds; but the reaction time can be longer, for example from about 30 seconds to about 5 minutes, from about 3 seconds to about 3 minutes, or from about 30 seconds to about 2 minutes.

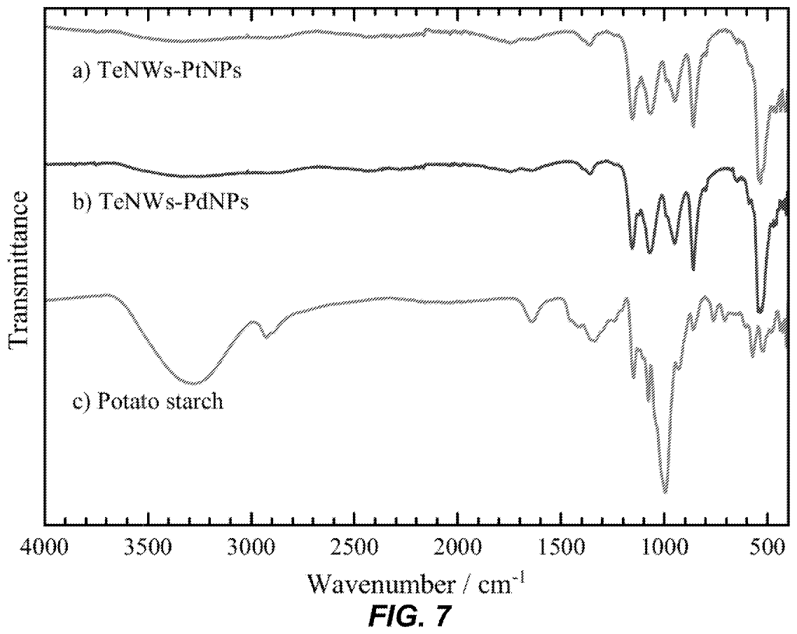

The FT-IR spectra (acquired in ATR mode) of samples PtNPs-TeNWs, PdNPs-TeNWs, and potato starch are compared in FIG. 7. In each of the TeNWs-based samples, the very weak and broad vibrational band at around 3300 cm⁻¹ and the medium vibrational signals found in the region between 800-1150 cm⁻¹ were assigned to the O—H stretching and glycosidic linkage, respectively (Fang et al. 2002; Ramazan Kizil, Joseph Irudayaraj, and Seetharaman 2002). The origin of these signals can be related to the starch-functionalized GREEN-TeNWs. In the case of PdNPs-TeNWs, the strong peak at 534 cm⁻¹ was related to the Pd—O bond (Tura et al. 1988), while in PtNPs-TeNWs, the strong peak at 545 cm⁻¹ was assigned to the vibration of the Pt—O bond (Khokhar, Xu, and Siddik 1994). As discussed below, these findings suggest the synthesis of oxide nanoparticles of both metallic elements, platinum oxide and palladium oxide nanoparticles.

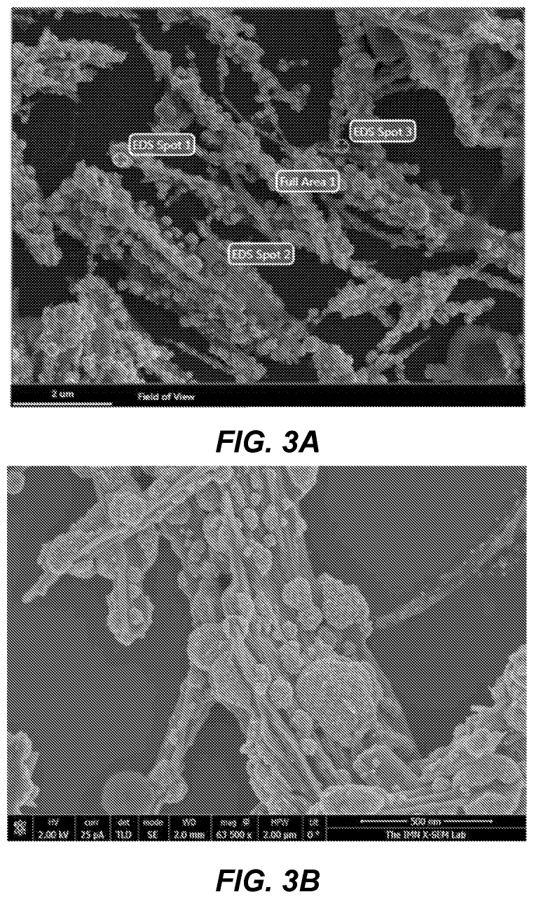

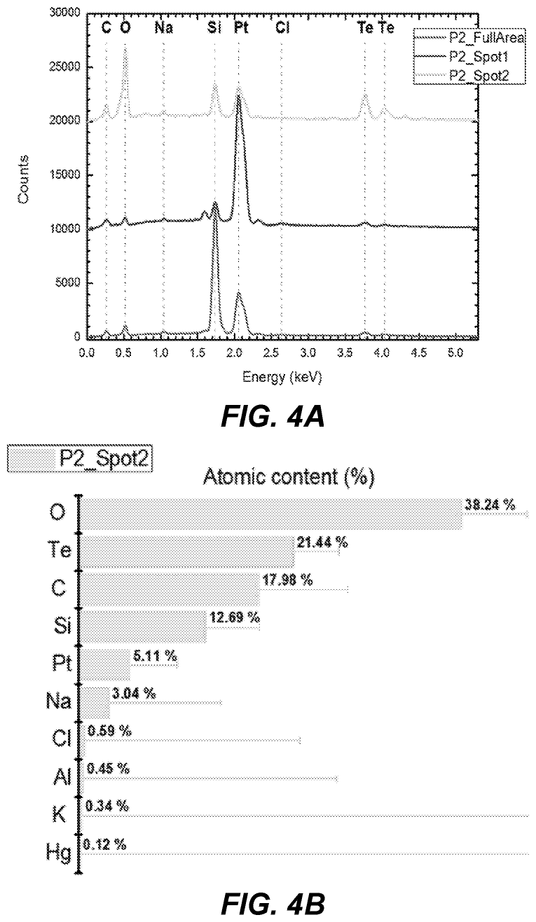

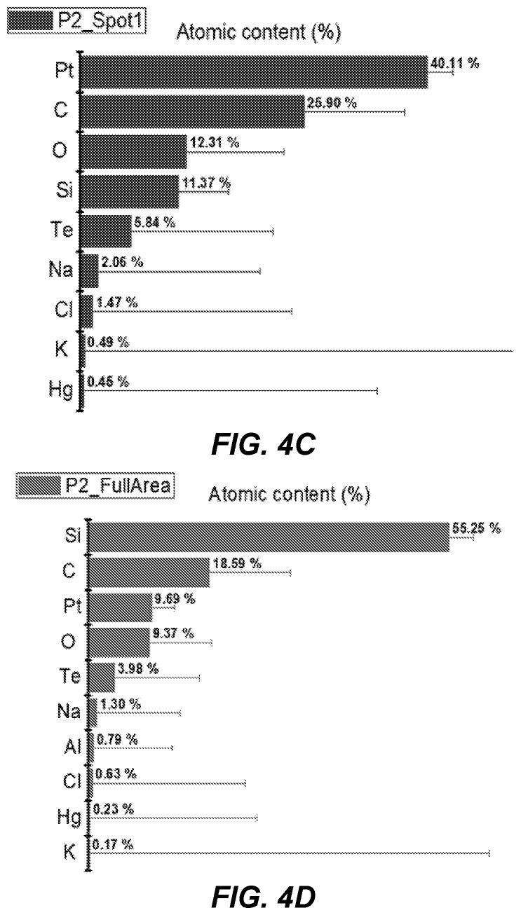

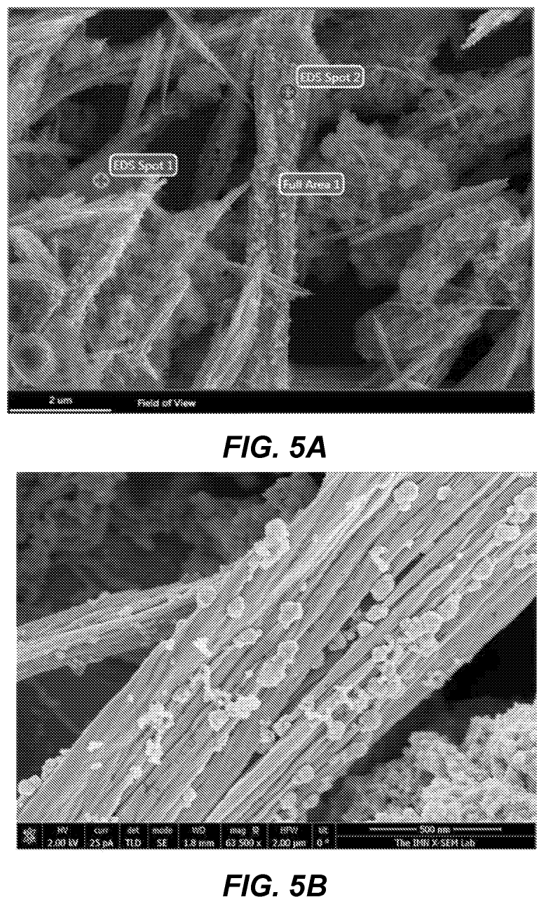

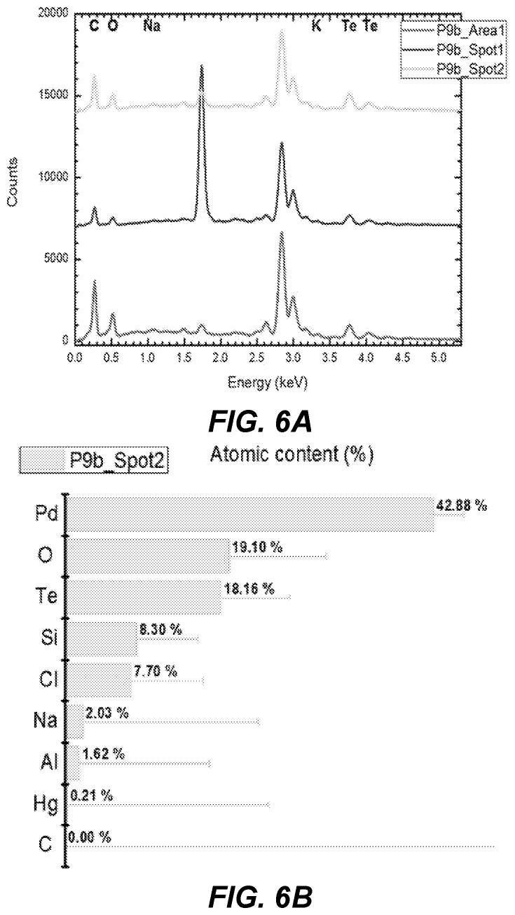

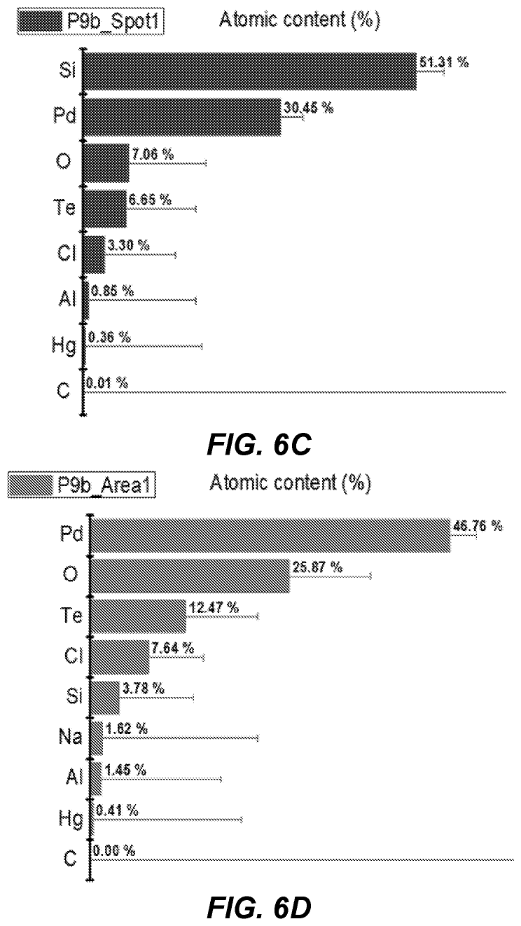

To further understand the composition of the synergistic structure of TeNWs and nanoparticles, energy-dispersive X-ray spectroscopy (EDS or EDX) analysis is measured on different points on the sample. FIG. 3A shows the presence of Pt nanoparticles all over the surface of the nanowire, and EDS analysis spots are shown in FIG. 3A. In FIG. 3A, spot 1 focuses on the nanowire structure and spot 2 is centered on the metallic nanoparticle, therefore the composition between them mismatch. FIG. 4B shows the atomic (%) composition of spot 2, and FIG. 4C shows the atomic (%) composition of spot 1. Atomic (%) composition for the full area region annotated in FIG. 3A is shown in FIG. 4D. In general, it can be said that high quantities of platinum and tellurium are present coming from the nano-synergetic structure. Moreover, carbon and oxygen peaks can be associated to residual starch, which is present on the initial GREEN-TeNWs and now covering the surface of the metal nanostructures. Similarly, FIG. 5A shows Pd nanoparticles growing on the top of the Te nanowire with EDS spots for analysis indicated in FIG. 5A. Again, tellurium and palladium peaks in FIGS. 6A-6D confirm the synergetic structure. Even though different spots of the sample were analyzed (see FIG. 5A), high amounts of Pd is found all over the sample. It is hypothesized that, due to the lower power of reduction of Pd, in comparison with Pt, more nanoparticles are formed. Hence, less content of carbon and oxygen, previously discussed as related with the starch-coating, is found as it is used in the reduction process. Besides, and further discussed in the XPS and XRD analyses, the high amount of oxygen present on the sample can be related with the partial incorporation of it into the metallic structure making, instead of Pd and Pt nanoparticles, PdO and PtO nanoparticles. Other elements present on the sample, such as silicon, come from the support used for the imaging. Other peaks such as sodium or chloride peaks might come from residual salts in water.

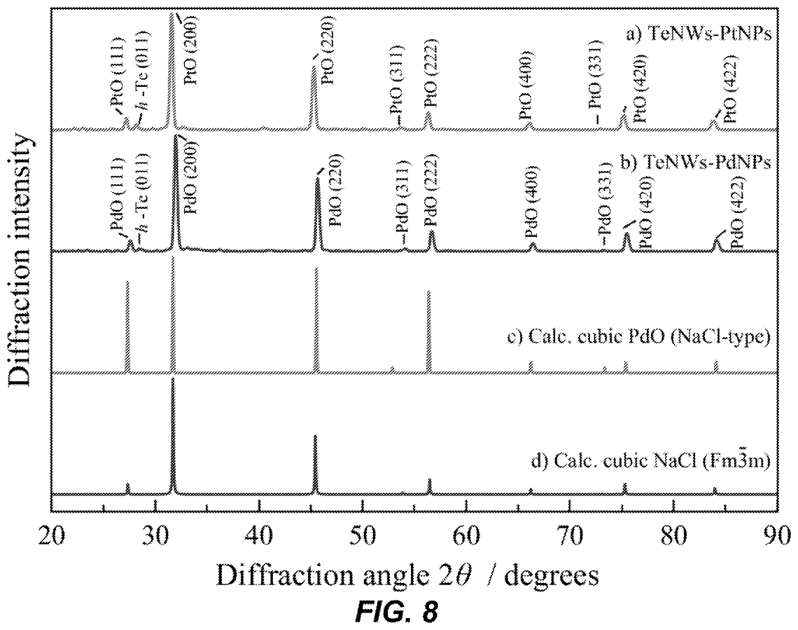

In FIG. 8, the experimental XRD patterns of the PtNPs-TeNWs and PdNPs-TeNWs are compared with the calculated XRD patterns of cubic PdO with NaCl-type structure (Kumar and Saxena 1989) and NaCl (space group Fm3̅m, Walker et al. 2004). Miller indices for cubic PtO and PdO NaCl-type structures are annoted in the PtNPs-TeNWs (top trace) and PdNPs-TeNWs (2^{nd} from top trace) spectra, respectively. Both experimental diffraction patterns can be principally indexed to their corresponding metal oxides, i.e. cubic PtO and PdO with NaCl-type structures for PtNPs-TeNWs and PdNPs-TeNWs, respectively. The lattice parameter (a) calculated for the cubic TeNWs-based Pt nanostructures was a=5.665±0.007 Å, which is in agreement with the reported values for a cubic PtO NaCl-type (a=5.65±0.05 Å). In the case of the sample PdNPs-TeNWs, the calculated lattice parameter was a=5.613±0.015 Å, which deviates significantly (around 9%) from the reported values for a cubic PdO NaCl-type (a=5.15±0.05 Å, Kumar and Saxena, 1989). Interestingly, in both experimental diffraction patterns (FIG. 8), the relative low intensity diffraction peak at 2θ=28.21° may be indexed to the (101) plane of elemental hexagonal Te in the sample, in alignment with the non-amorphous crystal structure of the GREEN-TeNWs. Moreover, the presence of further low intensity peaks in both experimental XRD patterns may suggest the minor presence of crystalline phases related to bimetallic Pt/Te (Bhan, Gödecke, and Schubert, 1969) and Pd/Te structures (Wopersnow and Schubert, 1977), for PtNPs-TeNWs and PdNPs-TeNWs, respectively.

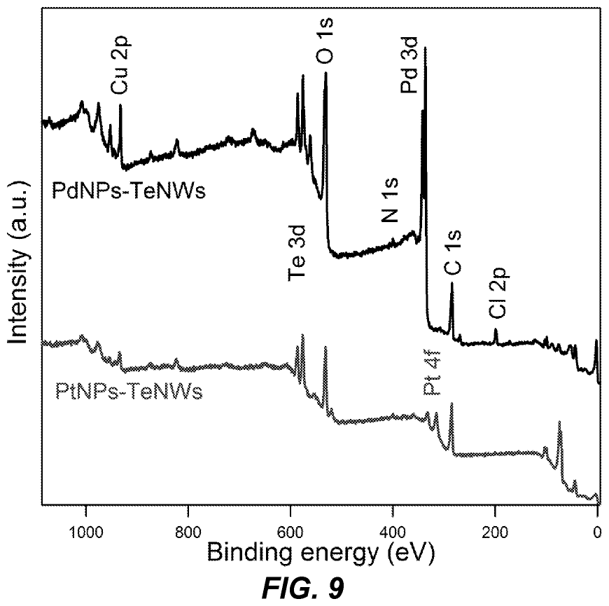

XPS analysis is used to characterize the chemical composition and electronic states of the PtNPs-TeNWs and PdNPs-TeNWs. FIG. 9 displays the wide scans that have been normalized to the C 1s core level peak for the sake of comparison. Table 1 presents the composition of the samples extracted from the analysis of the wide energy range scans.

TABLE 1
Composition of the samples extracted
from the wide energy range scans.

| | Composition % atomic | | | | | | |
|---|---|---|---|---|---|---|---|
| | O | C | N | Te | Pd | Pt | Cl |
| PdNPs- | 48.5 | 31.8 | 1.6 | 2.7 | 12.4 | — | 3.0 |
| TeNWs | | | | | | | |
| PtNPs- | 31.0 | 59.7 | 2.2 | 4.7 | — | 2.5 | 0.0 |
| TeNWs | | | | | | | |

In the two samples shown in Table 1, O, C, N, and Te are detectable. The amount of oxygen in the PdNPs-TeNWs is 1.5 times higher than in the PtNPs-TeNWs. In addition, the amount of Pd detected is approximately 5 times higher than the Pt in the other sample. These results were found in concordance with the information extracted from EDX and XRD analysis, suggesting that although the same concentration was used in both processes, the lower power of reduction for Pd allowed the generation of higher amount of nanoparticles. Moreover, the larger presence of Pd surrounding the TeNWs could be the reason for the smaller XPS signal of Te in this sample (nearly half of the signal for the PtNPs-TeNWs sample). Note that the PdNPs-TeNWs sample presents 3% of chlorine, probably arising from aqueous solvent. In addition, Cu signals from the substrate were also detected, but not considered in the quantification.

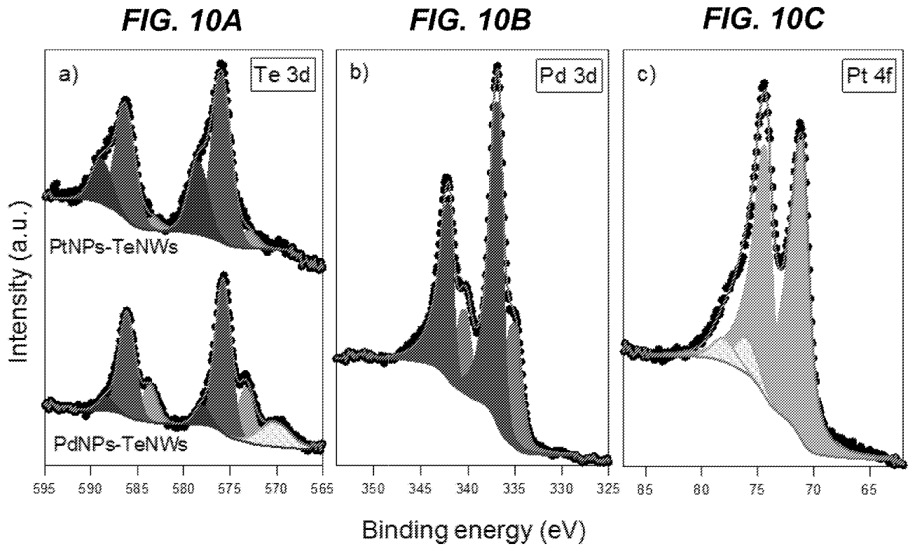

Detailed analysis of the core level peaks enables the observation of certain differences between the samples. FIG. 10A presents the Te 3d core level peak of the samples. Three components are found present. By referencing the values to the Te 3d_{5/2}, the first component at 573 eV corresponded to Te⁰. The second component at 575.7 eV corresponds to the oxidized component (Briggs 1981; Silva et al. 2016). A third and final component appeared at a binding energy of 578.0 eV. This energy, which is too high for standard Te compounds, was associated to Te(OH)₆ due to its tabulated BE (binding energy) at 577.1 eV. It has been hypothesized that this structure could come from the further reduction of the tellurium from the nanowires due to the presence of the coating, starch acting as reducing agent. From these analyses, it could be observed that the PdNPs-TeNWs presented larger proportion of non-oxidised Te, while the PtNPs-TeNWs sample presents more Te(OH)₆, as said beforehand, the higher power of reduction of Pt in comparison to Pd, may allow the starch to further reduce tellurium into the hydroxide structure instead of reducing the Pt.

Additionally, the component on the PdNPs-TeNWs sample at 570.2 eV (FIG. 10A, bottom trace) was related to the Cu_{LMM} auger peak from the substrate. The summary of the proportion of each component is shown in Table 2.

TABLE 2
Components extracted from the analysis of the Te 3d, Pd 3d,
O 1s and C 1s core level peaks of the PdTe and PtTe samples.

| | Te⁰ | TeOx | TeOH | Pd⁰ | PdOx | O1 | O2 | C—C/C—H | C—O | C═O | O_C═O | sp2 |
|---|---|---|---|---|---|---|---|---|---|---|---|---|
| PdTe | 15.5 | 43.4 | 41.1 | 32.1 | 67.9 | 39.3 | 60.7 | 32.3 | 22.9 | 12.2 | 5.4 | 27.1 |
| PtTe | 6.8 | 64.9 | 28.3 | — | — | 100 | — | 51.9 | 25.6 | 12.0 | 8.7 | 1.7 |

The analysis of the Pd 3d core level (FIG. 10B) presents two components: one at 335 eV that corresponds to the metallic component in the Pd 3d_{5/2}, while the other component at 336.8 eV arises from the oxide. From this analysis, it is clear that most of the Pd is oxidized (68%), therefore showing the formation of palladium oxide nanoparticles. On the contrary, the analysis of the Pt 4f core level of the PtTe sample (FIG. 10C) evidenced that all the Pt was only present in metallic form as reflects the component at 71 eV of the Pt 4f_{7/2}. A small component of oxide was found on the sample but without enough contribution to be considered. One explanation could come from the fact that Pt was found in low proportion than Pd in the top of the nanowires, hence if the structure is covered with an organic coating (e.g., starch) it is possible that the oxide compounds were not totally described with these techniques of characterization. In addition (FIG. 10C), the two small components at 76 and 78 eV corresponded to the Cu 3p.

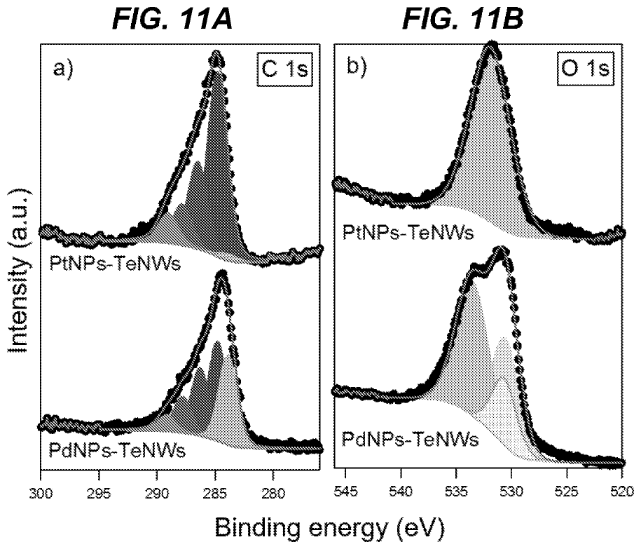

The analysis of the C 1s core level spectra of the samples revealed certain differences between the samples (FIG. 11A). Five components were used for the fitting. All of them were forced to have the same full width at half maximum (FWHM). These components correspond to C—C/C—H bonds at 285±0.2 eV; C—O/C—N bonds at 286.3±0.2 eV; C═O bonds at 288.1±0.2 eV; and O—C═O bonds at 289±0.2 eV. These four components are typically from organic compounds (Beamson, G.; Briggs 1993). The fifth component that appeared at a lower binding energy, BE, (284±0.2 eV) can be attributed to carbon in the sp² configuration (Nevshupa et al. 2011). The relative intensity of each component varies depending on the sample. Table 2 (above) displays the proportion of each component. A main difference between both samples relies in the abundance of the sp2 component, which is much higher in the PdNPs-TeNWs sample. The proportion of the oxidized compounds is similar in both samples. Mainly, the organic structure was related to the presence of starch coating both synergetic structures, which had been also found previously in the bare nanowire structure.

The O 1s core level of the samples presents significant differences (FIG. 11B), related with the oxide and hydroxide structures discussed beforehand. In the case of the PdNPs-TeNWs sample, two different contributions can be observed, one at 533.6 eV and another at 530.6 eV. The first component could be attributed to the organic compounds, while the second, at lower BE (binding energy), is related to the presence of palladium and tellurium oxides in the sample (at 530 eV and 530.8 eV, respectively), previously observed in the analysis of the Te 3d and Pd 3d core levels. The third component included in the fitting at 530. 6 eV corresponded to the Pd 3d that overlaps in this BE range. On the other hand, for PtNPs-TeNWs, the oxygen peak can be fitted with a single component at 531.4 eV, an intermediate energy which can include the oxidized organic compounds (usually around 532.3 eV) and the Te oxide (usually at 530.8 eV).

The characterization supports the nanoparticles contain palladium oxide and platinum oxide. In a standard chemical procedure for generation of these nanostructures, strong reducing agents, such as NaBH₄, sodium hydroxide (NaOH), sodium benzoate or trioctylphosphine, and additional stabilizing agents, like polynynylpolirridone (PVP), are needed. Besides, reaction conditions that are far away from standards of temperature and pressure are also needed, such as calcination at 500° C. under synthetic air conditions, and reflux conditions combined with microwave or an argon atmosphere set up. Surprisingly, the technology herein is an easy synthesis reaction at atmospheric pressure and room temperature, with no need of stirring or addition of reducing/capping agents using water as the unique solvent.

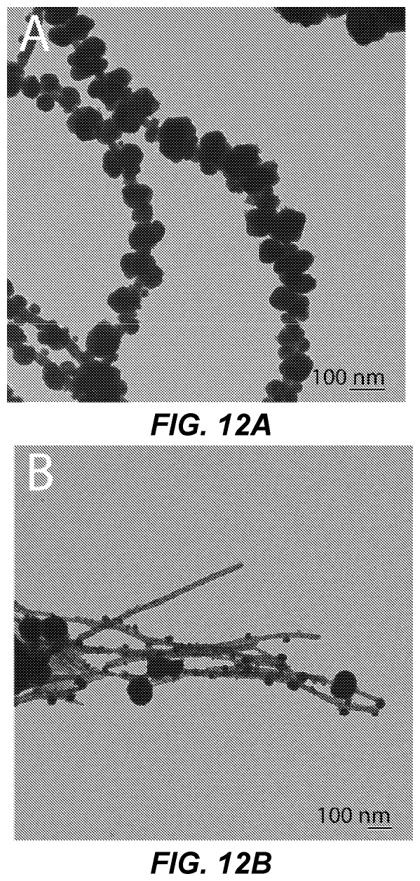

In order to verify the stability of the nanostructures, TEM imaging on the samples after 60 days of synthesis were carried out (FIGS. 12A and 12B). In general, it is evident that the samples kept their original morphologies and features. For example in FIG. 12A, the 60-days old PdNPs-TeNWs sample is composed of partial agglomerated thin nanoneedles (about 50-200 nm length or longer and about 2-15 nm wide). These features are in accordance with the freshly synthesized nanomaterials (FIG. 1A). Stability analysis through the measurement of the Z-potential of the freshly synthesized, 60-days old, and 120-days old Te-based nanomaterials was also carried out. In general, a colloid or suspension is considered stable if the Z-potential is near or above a critical value of ±30 mV. Given the measured Z-potential values for the colloids (freshly and 120-days old samples, see Table 3), they can be considered stable. In Table 3, the pH of the colloids was 7.0±0.2.

TABLE 3
Zeta-potential values for freshly and 120-days old palladium
and platinum NPs-TeNWs.

| | Z-potential (mV) | |
|---|---|---|
| Nanostructure | As-synthesized | 120 days-old |
| PdNPs-TeNWs | −27.21 ± 1.81 | −25.55 ± 1.49 |
| PtNPs-TeNWs | −28.02 ± 2.02 | −25.09 ± 1.33 |

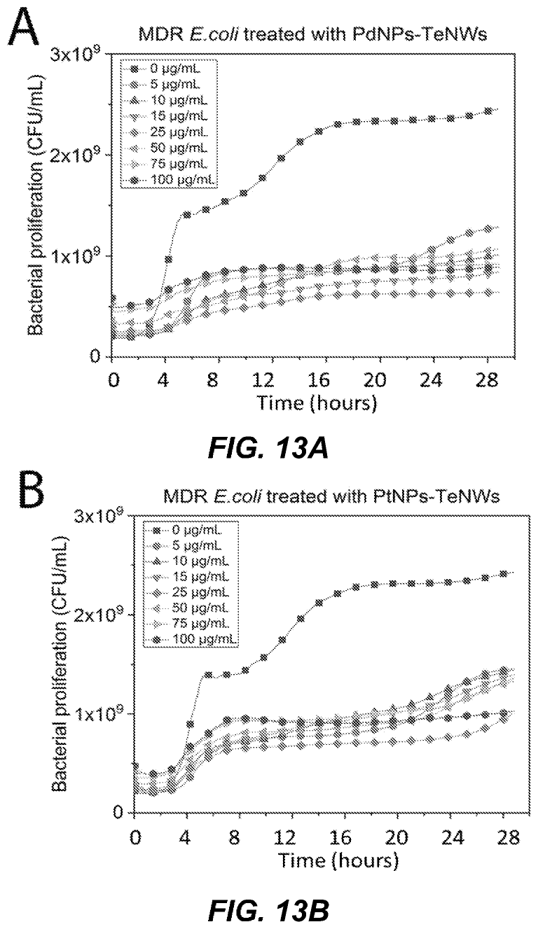

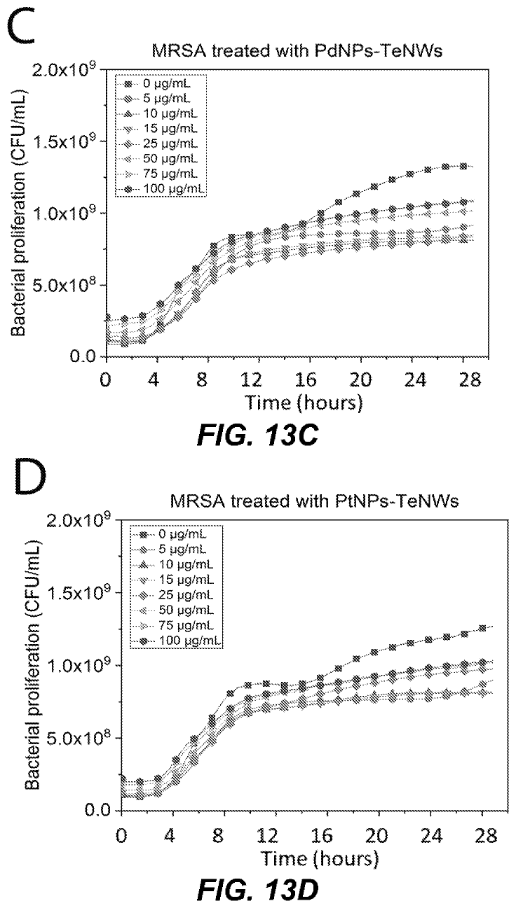

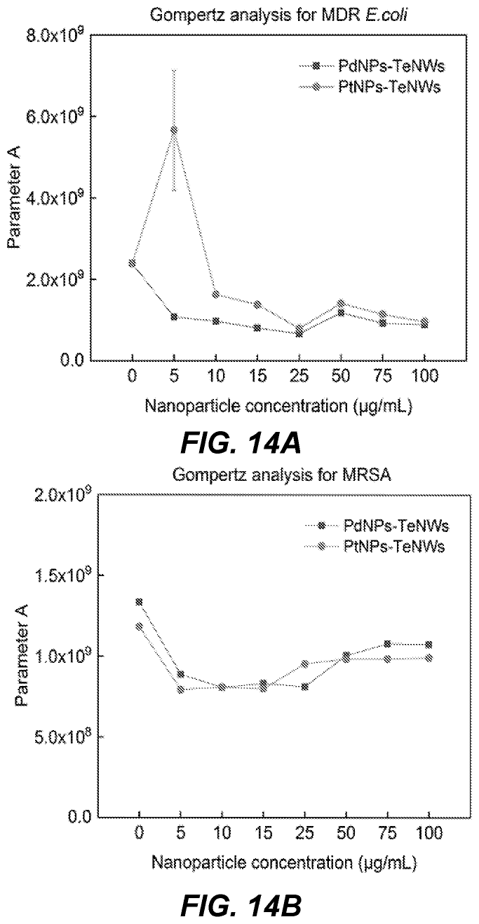

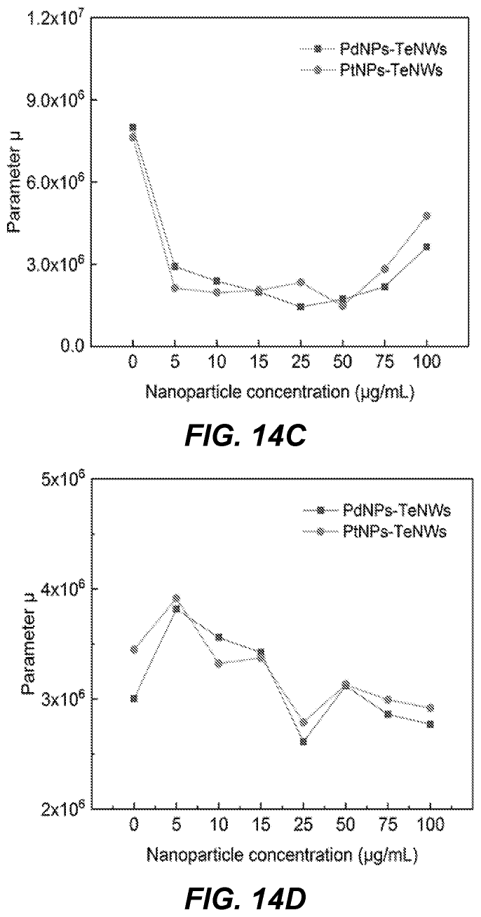

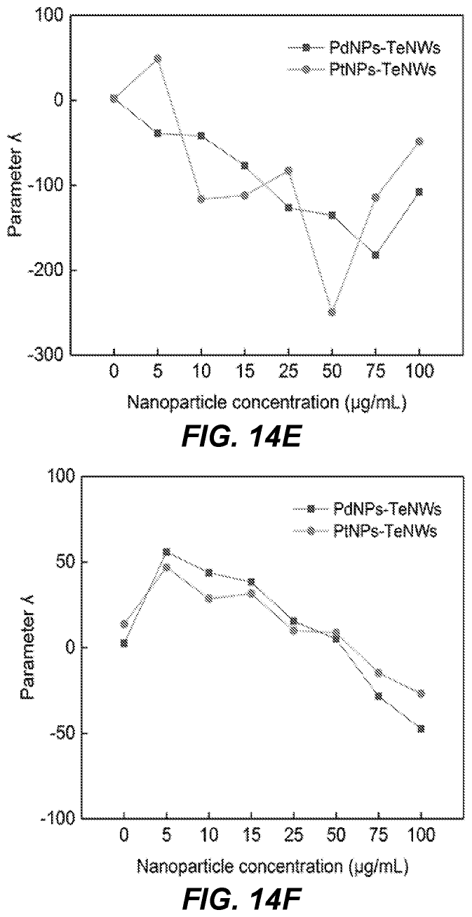

The antibacterial effect of the PdNPs- and PtNPs-TeNWs structures was first studied using 24 hours-growth curve analysis to observe the potential changes in the bacterial growth when cultured with different concentrations of the nanomaterials. Nanoparticle concentrations between 5 and 100 μg/mL produced a delay in the growth of MDR E. coli (FIGS. 13A-13B) for both entities, with a relative dose-dependent inhibition. On the other hand with MRSA (FIGS. 13C-13D), the concentration of the nanostructures seems not to have as an important effect in the bacterial growth, with a slight inhibition at the end of the exponential phase. To further investigate the effects of the nanostructures on the bacterial growth, the parameters of the modified Gompertz equation (Equation 2 below) were calculated and plotted for analysis (FIGS. 14A-14F). In Equation 2, the parameter A, which represents the maximum bacterial growth, decreased at increasing concentrations (FIGS. 14A-14B), a behavior that was specially visible in experiments with MDR E. coli. On the other hand, changes in the maximum bacterial growth rate was determined by analyzing the parameter p. This analysis demonstrated that higher nanostructures concentrations resulted in an overall lower growth rate of the bacteria (FIGS. 14C-14D). The decay was specially visible in experiments with MDR E. coli even at the minimum concentration. Lastly, the parameter k, which represents the lag time in the bacterial growth, or the duration of time where bacteria are adapting themselves to the growth conditions offered by the media, was analyzed (FIGS. 14E-14F). This analysis showed that higher nanostructure concentrations led to a shorter lag phase in bacterial growth. This was especially visible with MDR Escherichia coli (FIG. 14E). This suggests that the presence of both PdNPs- and PtNPs-TeNWs delays bacterial maturation, therefore inhibiting bacterial growth.

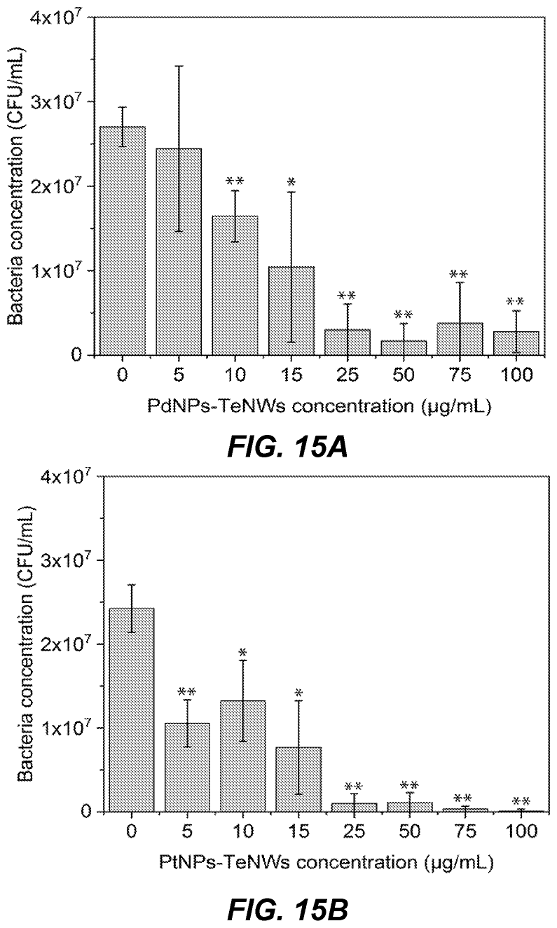

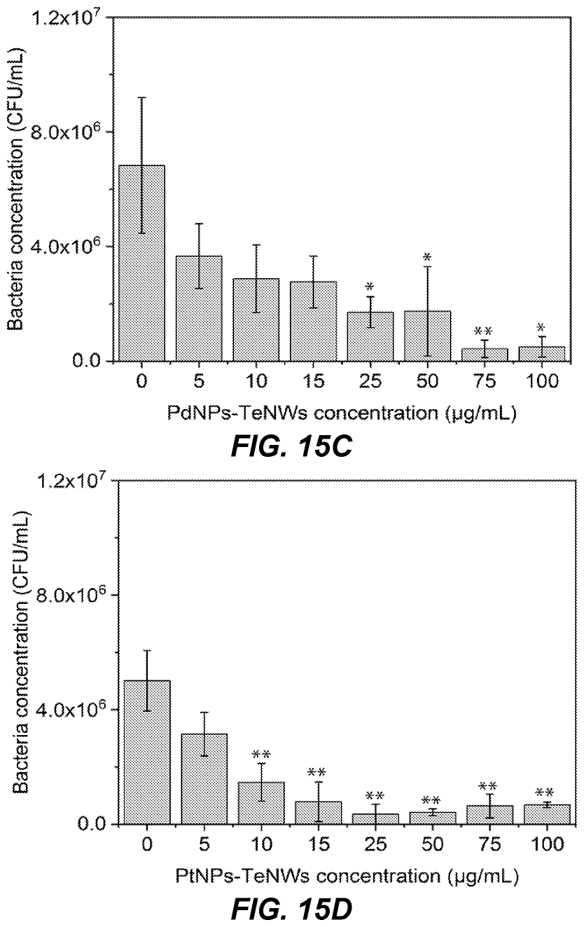

Colony counting unit assay conducted over MDR E. coli (FIGS. 15A, 15B) and MRSA (FIGS. 15C, 15D) showed a dose-dependent inhibition of the bacterial growth when exposed to different concentrations of both PdNP- and PtNP-TeNWs. The palladium containing nanostructures were effective towards MDR E. coli at a range of concentrations between 10 and 100 ug/mL, while a concentration range between 25 and 100 ug/mL showed to be effective towards MRSA. On the other hand, PtNP-TeNWs produced a delay in MDR E. coli proliferation in a range of concentrations up to 100 ug/mL, while a concentration range between 10 and 100 ug/mL was successful inhibiting the proliferation of MRSA. Therefore, a wide range of concentrations was shown to be effective in both antibiotic-resistant phenotypes. MIC values were calculated as an extension of the antibacterial behavior.

TABLE 4
MIC values for different nanoparticles against
MDR E. coli and MRSA.

| MIC values (μg/mL) | MDR-Escherichia coli | MRSA |
|---|---|---|
| PdNPs-TeNWs | 24.79 | 26.45 |
| PtNPs-TeNWs | 15.24 | 15.86 |

[[These values differ from others found in literature, showing either a decrease or similitudes of the MIC values for both nanosystems. For example, Tahir et al. showed that PdNPs produced by the Sapium sebiferum, tested against Staphylococcus aureus, rendered a MIC of 45.4 μg/mL (Tahir et al. 2016), while Dhanavel et al. reported the MIC of chitosan supported PdNPs towards Escherichia coli, with a value of 25 μg/mL (Dhanavel et al. 2018). On the other hand, Khan et al. reported the synthesis of PtNPs using pectin and sodium borohydride which were tested against Escherichia coli, with a MIC around 12 μg/mL (Ayaz Ahmed, Raman, and Anbazhagan 2016), while PtNPs prepared from marine actinobacteria (Streptomyces sp.). showed a MIC of 20 μg/mL when tested towards the activity of Staphylococcus aureus (Dev Sharma 2017).]]

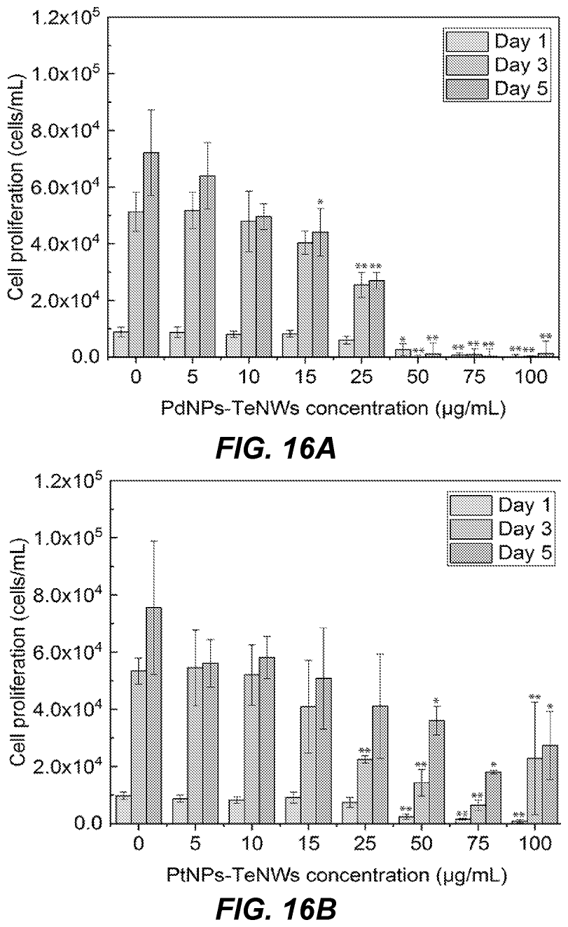

The effects of the nanomaterials towards human cells is studied. A dose-dependent cell proliferation decay was found when the two nanosystems were cultured with HDF cells over a period of time of 5 days (FIGS. 16A-16B). For PdNP-TeNWs in FIG. 16A, a low cytotoxic effect was found in a range of concentrations between 5 and 25 ug/mL at 24 hours, while the range was reduced at concentrations up to 10 ug/mL at the fifth day. When PtNP-TeNWs were present in the cell media (FIG. 16B), the optimum range of concentrations was found to be 5 to 15 ug/mL in experiments up to 5 days. Therefore, the PdNP-TeNWs and PtNP-TeNWs can be considered biocompatible in a range of concentrations up to 10 and 15 ug/mL respectively.

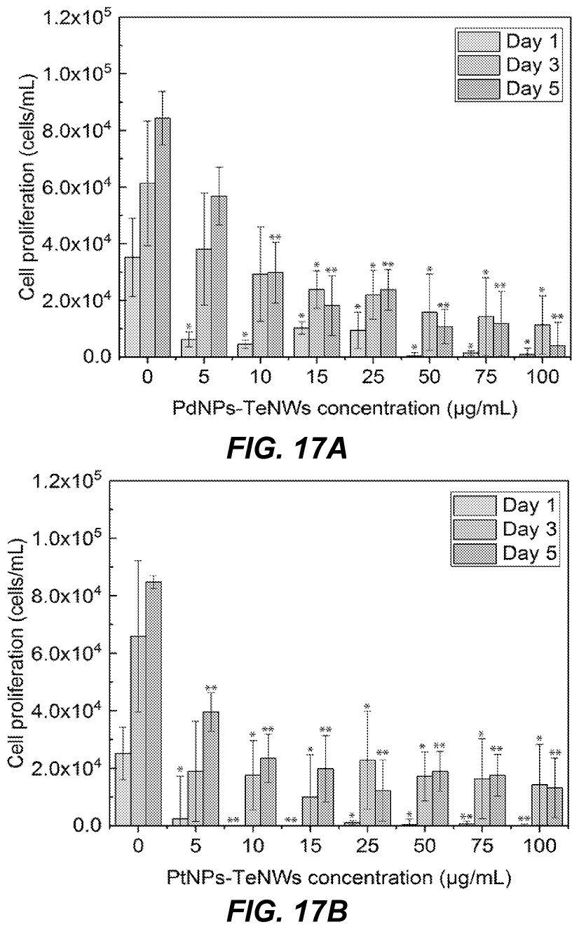

Moreover, a dose-relative cell proliferation decay was found when both nanostructures were cultured with human melanoma cells for a period time of 3 days, and a dose-dependent cell proliferation decay was found when the both nanostructures were cultured with melanoma cells for five days (FIGS. 17A-17B). For PdNP-TeNWs, the anticancer effect towards melanoma cells was found in a range of concentrations up to 25 ug/mL at 24 hours with low cytotoxic effect, while the range was reduced at a concentration of only 15 ug/mL at the third day (FIG. 17A, Table 5 and 6 below). When PtNP-TeNws were present in the cell media, the optimum range of concentrations was found to be 5 to 15 ug/mL in experiments up to 5 days (FIG. 17B). Thus, the PdNP-TeNWs can be considered anticancer at the concentration of 15 ug/mL for a 3-day treatment and PtNP-TeNWs can be considered between 10-15 ug/mL for a 5-day treatment. IC₅₀ values were calculated to further study the response of the cells to the nanostructures.

TABLE 5
IC50 values for different nanoparticles cultured
with HDF cells.

| IC50 values | | | |
|---|---|---|---|
| (μg/mL) | 1 day | 3 days | 5 days |
| PdNPs-TeNWs | 21.25 | 17.64 | 38.75 |
| PtNPs-TeNWs | 30.46 | 24.37 | 35.85 |

TABLE 6
IC50 values for different nanoparticles cultured with
human melanoma cells.

| IC50 values | | | |
|---|---|---|---|
| (μg/mL) | 1 day | 3 days | 5 days |
| PdNPs-TeNWs | 8.00 | 26.55 | 30.74 |
| PtNPs-TeNWs | 7.57 | 10.04 | 12.45 |

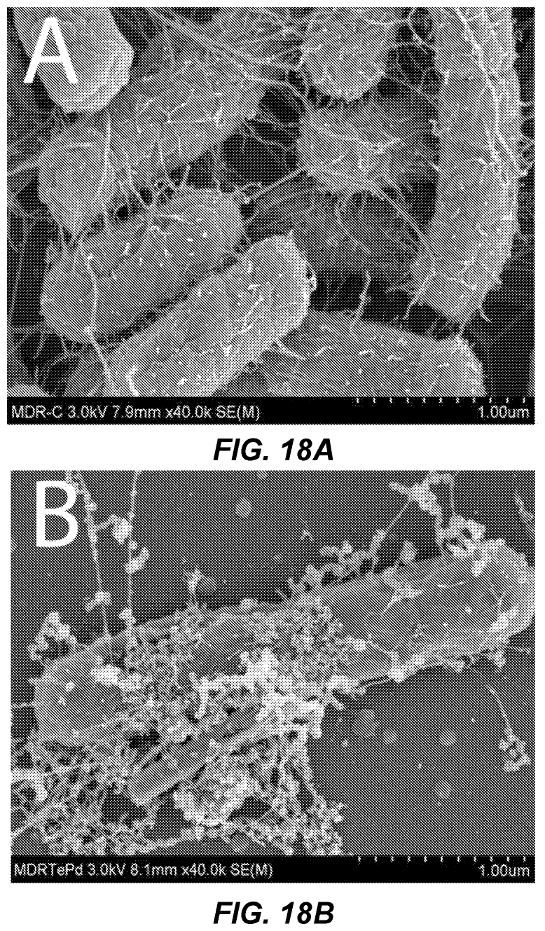

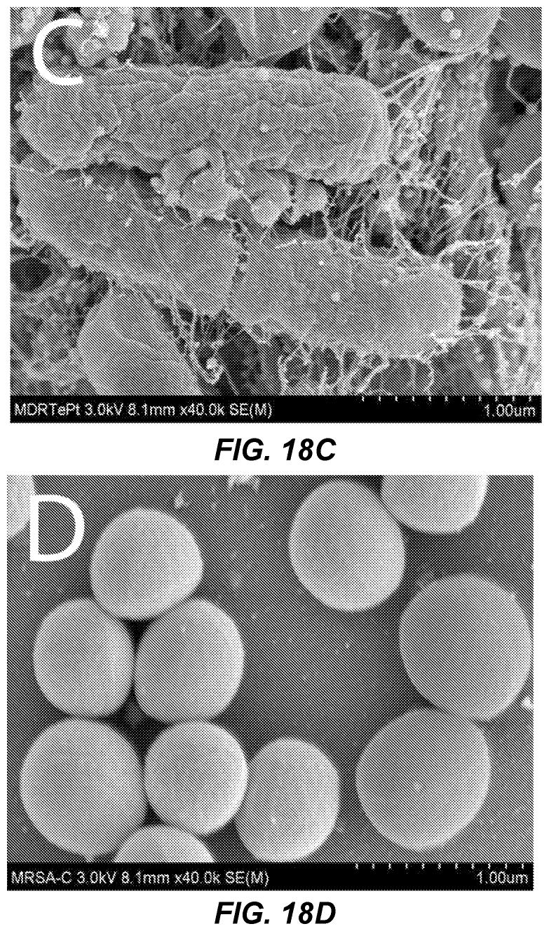

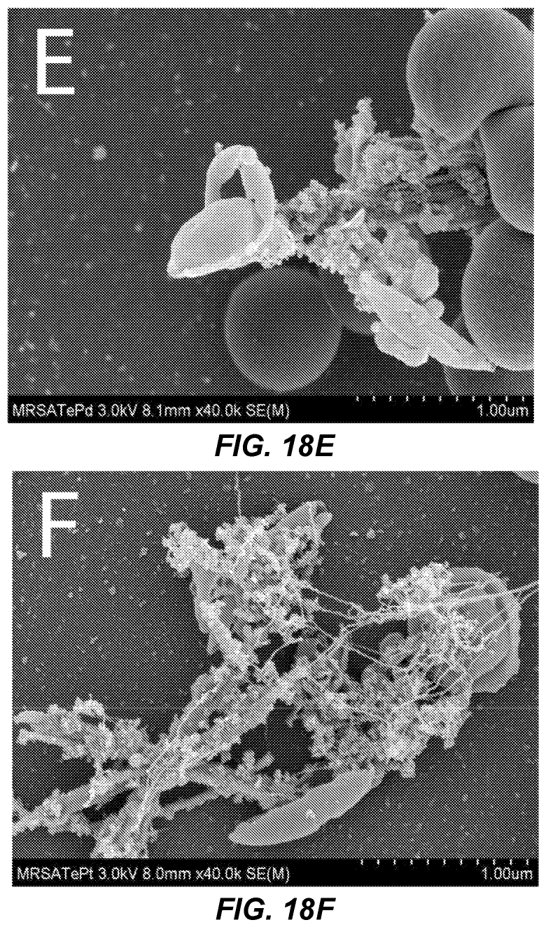

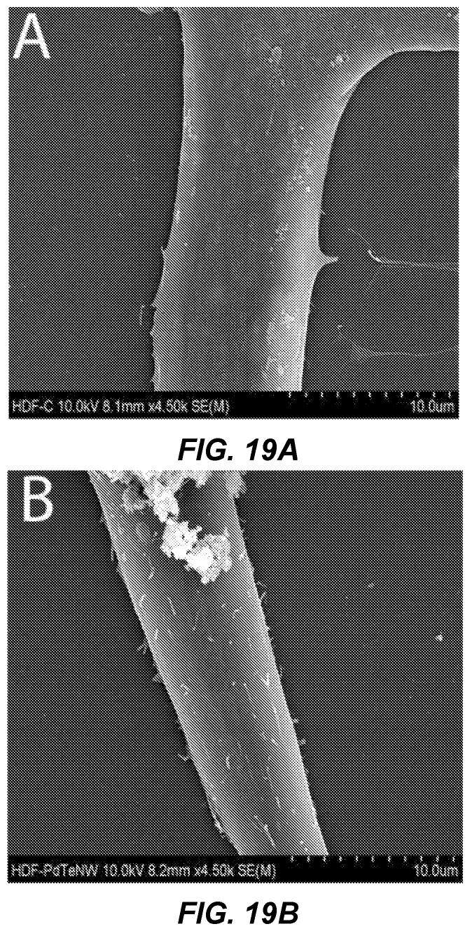

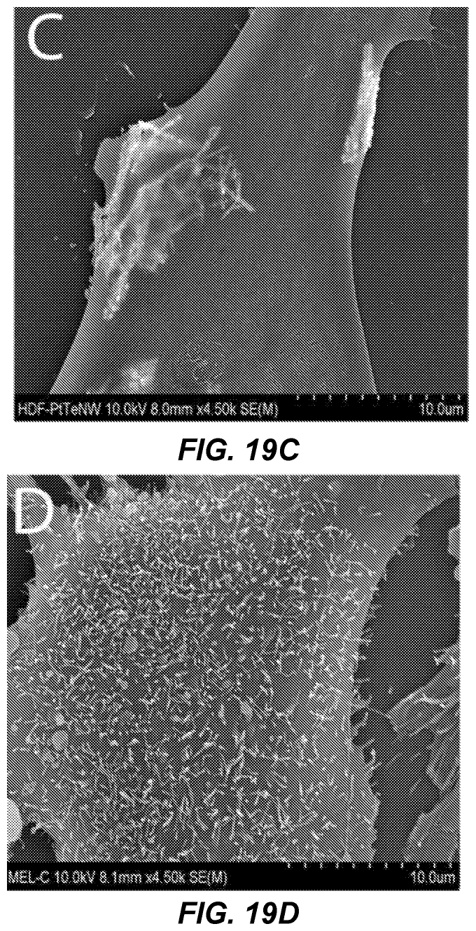

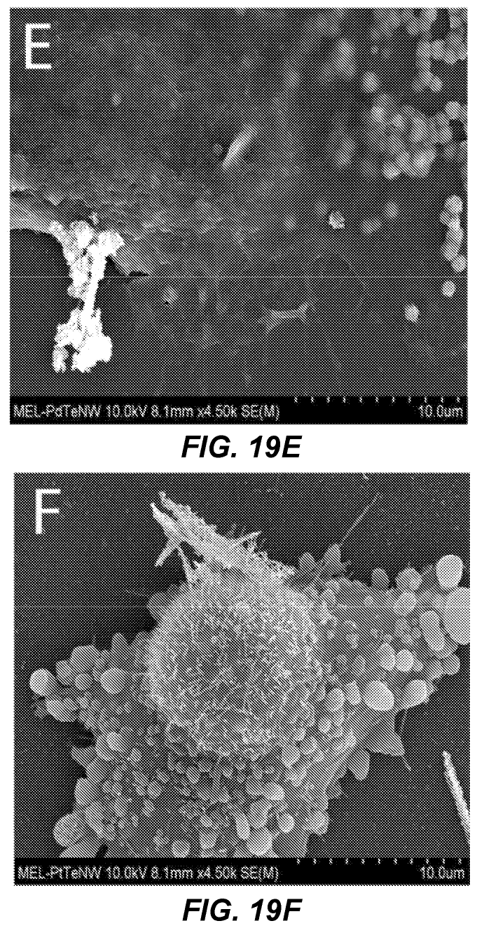

Cell fixation and SEM imaging for bacteria and human cells is shown in FIGS. 18A-19F. SEM micrographs of control MDR E. coli bacteria (FIG. 18A) and after treatment with PdNPs-TeNWs (FIG. 18B) and PtNPs-TeNWs (FIG. 18C) show significant changes in the bacteria. SEM micrographs of control MRSA (FIG. 18D) and MRSA bacteria after treatment with PdNPs-TeNWs (FIG. 18E) and PtNPs-TeNWs (FIG. 18F) also show significant changes in the bacteria. The characterization indicates that the treatment with the synergetic structures induces changes in both bacterial strains. Disruption of the outer cell membrane and cell lysis is seen after the treatment with both nanostructures. Therefore, clear cell damage is observed, with abundant presence of holes and cracks all over the cell membrane, and bacterial deformation and collapse. The cell membrane damage is commonly found to be a cause from reactive oxygen species (ROS). Nevertheless, other mechanisms can also be inferred, for example, the direct damage of the cells due to the morphology of the nanostructures. From the SEM images of the bacteria, we can see that the membrane damage occurs and that there is attachment of nanoparticles to bacteria.

SEM micrographs were obtained of HDF and melanoma cells with no nanoparticle treatment (FIG. 19A, FIG. 19D) and after treatment with PdNPs-TeNWs (FIG. 19B, FIG. 19E) and PtNPs-TeNWs (FIG. 19C, FIG. 19F). The untreated HDF cells shown in FIG. 19A are compared to the PdNPs-TeNWs treated HDF cells in FIG. 19B and the PtNPs-TeNWs treated HDF cells in FIG. 19C. As can be seen, HDF cells were able to successfully proliferate in the presence of the nanostructures, that were either deposited on top of the cell membrane or underneath the structure, with no apparent disruption or alteration of membrane or normal growth. On the other hand, the presence of the nanostructures induced a severe presence of bubbles and membrane disruption within the melanoma cell population (see FIGS. 19E-19F). The untreated human melanoma cells are shown in FIG. 19D. Melanoma cells treated with PdNPs-TeNWs are shown in FIG. 19E, and melanoma cells treated with PtNPs-TeNWs are shown in FIG. 19F.

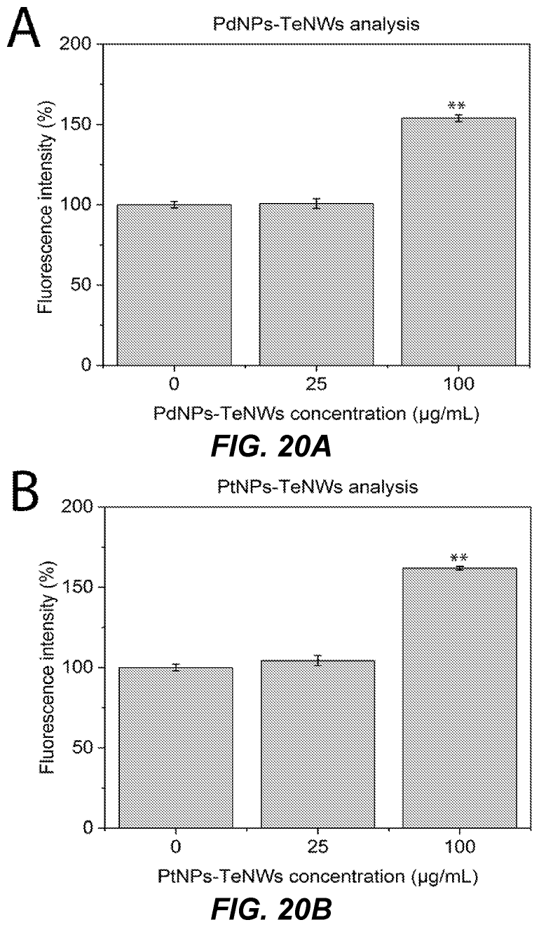

ROS studies are presented in order to explore the mechanism of NPs-TeNWs toxicity towards human melanoma cells; the production of ROS was evaluated in response to the exposure of two different concentrations of PdNPs-TeNWs and PtNPs-TeNWs: 25 and 100 μg/mL. Melanoma cells were exposed for 24 hours to the concentrations of NPs-TeNWs and ROS were quantified. Both nanostructures showed a similar production of ROS, with a slightly bigger release of species for PtNPs-TeNWs (FIG. 20B) compared to PdNPs-TeNWs (FIG. 20A). The contribution of ROS might be related to both tellurium and the noble metal presented on the structure, either palladium or platinum. Tellurium oxyanions have been found to trigger the generation of ROS, with the ability to react with intracellular thiols and forming intermediates that cause oxidative stress as a consequence of the formation of superoxide radicals.

Since the ROS generation is similar for both structures, but different results were obtained for the cytotoxicity analysis, there must be other mechanisms responsible for the anticancer activity of these nanostructured materials. Some proposed mechanisms are related to the nanostructures themselves, contributing to the cell damage by disrupting the integrity of the envelope; or to the surface chemistry and features of the nanostructures.

Surprisingly, green synthesized starch-mediated TeNWs were successfully used as nanometric templates for the in situ generation of noble PdNPs and PtNPs in a quick, environmentally-friendly, and cost-effective reaction with no need of additional reducing or capping agent. The nanoparticles, comprised of palladium and platinum oxide, were extensively characterized in terms of composition and surface chemistry. The structures were tested as biomedical agents. The PdNPs-TeNWs and PtNPs-TeNWs showed antibacterial properties in a range of concentrations between 10 and 25 ug/mL, triggering no cytotoxicity towards healthy epithelial cells over the same period of time. Furthermore, both nanostructures were found to be anticancer towards melanoma cells in a range of concentrations between 10 and 15 ug/mL with no alteration of the normal proliferation of healthy skin cells. Therefore, it is concluded that the technology can be successfully used at low concentrations as biomedical agents with antibacterial and anticancer properties, being biocompatible in the same range of concentrations.

### EXAMPLES

### Synthesis of the Synergetic Structures of PtNPs-TeNWs and PdNPs-TeNWs

The tellurium nanowire (TeNWs) template was synthetized with the following example green-synthesis protocol. Telluric acid (H₂TeO₄) was mixed with 0.15 g of a starch solution in deionized water. Then, the mixture was transferred into a Teflon-lined stainless-steel reactor and placed into an oven at about 160° C. for about 15 hours. After the reaction, the mixture was allowed to cool down at room temperature. A non-limiting example of a green synthetic route was previously shown in Reaction 1 above.

The GREEN-TeNWs were used as unique reducing agents for the production of the nanoparticles. The ability of green-synthesized TeNWs as a template for the reduction of Pt and Pd ions to their elemental form was successfully accomplished upon addiction of the corresponding metallic salt into a mixture of previously synthesized TeNWs and water. Briefly, a solution 1:1:15 in volume of newly synthesized TeNWs, 5 mg/mL of metallic salt precursor, and distilled water was prepared. The metallic precursors employed were palladium chloride (PdCl₂) (Sigma Aldrich, St. Louis, Mo.) and potassium tetrachloroplatinate (K₂PtCl₄) (Sigma Aldrich, St. Louis, Mo.) for the preparation of palladium and platinum nanoparticles, respectively. Then, the mixture was allowed to react for about 1 minute, with no heat or agitation. The reaction was conducted at room temperature, with no stirring and no need of additional reducing or capping agent. Right after, the volume was transferred and centrifuged and washed twice with distilled water at 10000 rpm for 20 min. A final pellet was collected from the bottom of the centrifuge tube and suspended in distilled water. The solution was lyophilized overnight, resulting in a black powder that was resuspended in the desired amount of water (for desired concentrations) for further characterization and experiments.

The role of starch in the reduction and capping of the nanoparticles was clear, since characterization through TEM before and after the reaction revealed the disappearance of the starch coating surrounding the nanowires. The hydroxyl groups of starch possibly facilitated palladium and platinum ions in solution to be reduced on the surface of the nanowires by electrostatic binding in the helical structure of amylose chains. As a consequence, a part of starch granules is converted to glucose that has an aldehyde group. The aldehyde functional terminal allows glucose to act as the reducing sugar, which can reduce the ions and trigger the generation of small nuclei all over the nanowires that will give rise to the nanoparticles. Thus, it was hypothesized that both Pd- and PtNPs can be formed by a simultaneous in situ reduction without the addition of any other reducing agent. Besides, the tellurium itself might be involved in the reduction. However, no reports of this behavior were found in literature.

### Stability Analysis

In order to analyze the stability of the samples, TEM and Zeta-potential measurements were completed in fresh, 60, and 120-days old PdNPs-TeNWs and PtNPs-TeNWs.

### Preparation of the Bacterial Cultures

Two bacterial strains that are resistant to antibiotics were employed for the study: Multidrug-resistant (MDR) Escherichia coli (ATCC BAA-2471; ATCC, Manassas, Va.) and Methicillin-resistant Staphylococcus aureus (MRSA) (ATCC 4330; ATCC, Manassas, Va.). These strains were selected for the antimicrobial tests to determine the effect of both PtNPs-TeNWs and PdNPs-TeNWs on the microbial growth. The cultures were maintained on agar plates at 4° C. Bacteria were inoculated into 5 mL of sterile Tryptic Soy Broth (TSB, Sigma) in a 50 mL Falcon conical centrifuge tube and incubated at 37° C./200 rpm for 24 hours. The optical density was then measured at 600 nm (OD600) using a spectrophotometer. The overnight suspension was diluted to a final bacterial concentration of 10⁶ colony forming units per milliliter (CFU·mL⁻¹) prior to measuring the optical density.

### Testing the Antimicrobial Effect of the Nanostructures

A colony of each bacterial strain was re-suspended in TSB (Tryptic Soy Broth) media and then placed in a shaking incubator to grow overnight remaining at constant 200 rpm and 37° C. The overnight suspension was diluted to a bacterial concentration of 10⁶ colony forming units per milliliter (CFU·mL⁻¹) and optical density measurements at 600 nm (OD600) were performed using a spectrophotometer. Furthermore, the seeding density was determined in each experiment using a colony forming unit assay. Different concentrations of both synergetic structures, PdNPs-TeNWs and PtNPs-TeNWs, were mixed with 100 μL of the different bacteria in TSB medium and added to each well of a 96-well plate for the specific antimicrobial assay (Thermo Fisher Scientific, Waltham, Mass.). For the control group, the bacteria was mixed with 100 μL of TSB culture media without the presence of any nanosystem (PdNPs-TeNWs or PtNPs-TeNWs), reaching a final volume of 200 μL per well. After the plate was completely prepared, the absorbance values of all samples were measured at 600 nm every 2 minutes on the absorbance plate reader for about 24 hours. The absorbance values related with the synergetic nanostructures were measured by preparing negative controls made by mixing of TSB medium and nanostructures only. For the conversion of OD to CFU/mL, standard curves were used for each type of the bacteria.

The bacterial growth curves were obtained and fitted into the Gompertz model (Zwietering et al., Applied and Environmental Microbiology, 1990) by subtracting the initial values to the entire curve and shifting them to the starting point. For the application of Gompertz distribution, re-parametrization was needed in order to describe the biological parameters (A, μ, and λ) (Equation 2) into mathematical ones (a, b, c . . . ) (Equation 1). The estimation of initial values in addition to the intervals of 95% confidence were difficult to calculate as it is not directly estimated into the equation.

y=a*e^{-e}^{^{(b-ct)}}

Equation 1. Gompertz equation in terms of mathematical parameters.

The Gompertz equation in terms of mathematical parameters was modified through a series of derivations to obtain the modified equation that was used for the fitting of the curves. The resulting equation describes a sigmoidal growth curve.

$$y = A * {e}^{- {e}^{\frac{μ \cdot e}{A} ( λ - t ) + 1}}$$

Equation 2. Gompertz equation in terms of biological parameters.

Where the parameter y is related to the number of bacteria (corresponding to the optical density reading), A is the maximal possible value of y, μ is the maximal growth rate, and λ is the lag time. The parameters A, μ and λ were estimated according to a least-squares estimation algorithm using a GRG (generalized reduced gradient) nonlinear solver.

The colony counting assays were done by seeding the bacteria in a 96-well plate and adding different concentrations of the synergetic structure. The plates were incubated at 37° C. during 8 hours and, after that period of time, the plates were removed from the incubator and diluted with PBS in a series of vials until a concentration diluted ×10⁵ and ×10⁶ times. Three drops of 10 μL were taken of each dilution and deposited in a TSB-Agar plate. After a final period of incubation of between about 8-10 hours at 37° C., or about 24 hours at 37° C., the numbers of colonies formed were counted at the end of the incubation.

### Testing the Effect of the Nanomaterials Towards Human Cells

Cytotoxicity assays were performed with primary human dermal fibroblasts (TCC® PCS-201-012™, Manassas, Va.) and melanoma (ATCC® CRL-1619, Manassas, Va.) cells. Cells were cultured in Dulbecco's Modified Eagle Medium (DMEM; Thermo Fisher Scientific, Waltham, Mass.), supplemented with 10% fetal bovine serum (FBS; ATCC® 30-2020™, American Type Culture Collection, Manassas, Va.) and 1% penicillin/streptomycin (Thermo Fisher Scientific, Waltham, Mass.). MTS assays (CellTiter 96© AQueous One Solution Cell Proliferation Assay, Promega, Madison, Wis.) were carried out to assess cytotoxicity. Cells were seeded onto tissue-culture-treated 96-well plates (Thermo Fisher Scientific, Waltham, Mass.) at a final concentration of 5000 cells per well in 100 μL of cell medium. After an incubation period of 24 hours at 37° C. in a humidified incubator with 5% carbon dioxide (CO₂), the culture medium was replaced with 100 μL of fresh cell medium containing concentrations from 5 to 100 μg/mL of both synergetic nanostructures.

Cells were cultured for different periods of time in order to evaluate the performance and interaction of the nanoparticles with the cells. Biocompatibility assays were done during 1, 3 and 5 days at the same conditions and then washed with PBS. Afterwards, the medium was replaced with 100 μL of the MTS solution (prepared using a mixing ratio of 1:5 of MTS:Medium). Subsequently, the 96-well plate was incubated for 4 hours in the incubator to allow for a color change in the MTS solution. Finally, the absorbance was measured at 490 nm on an absorbance plate reader (SpectraMAX M3, Molecular Devices) for cell viability after exposure to the NPs-NWs concentration. Cell viability was calculated by dividing the average absorbance obtained for each sample by the one achieved by the control sample, and then multiplied by 100. Controls containing cells and media, and just media, were also included in the 96-well plate to identify the normal growth of cells without nanoparticles and to determine the absorbance of the media itself.

### Cell Fixation and SEM Imaging (for Bacteria and for Human Cells)

For the fixation of bacterial cells, both bacterial strains (MDR Escherichia coli and MRSA) were inoculated into 5 mL of sterile TSB media in a 50 mL Falcon conical centrifuge tube and incubated at 37° C./200 rpm for 24 hours. The optical density was then measured at 600 nm (OD600) using a spectrophotometer. The overnight suspension was diluted to a final bacterial concentration of 106 colony forming units per milliliter (CFU/mL) prior to measuring the optical density. A selected 75 μg/mL concentration of PdNPs-TeNWs and PtNPs-TeNWs was mixed with TSB media and bacterial solution in a 6-well plate with a glass coverslip attached to the bottom. The coverslips were pre-treated with polylysine to enhance cell adhesion right before the experiment. The plate was placed inside an incubator for 8 hours at 37° C.

For the fixation of primary human dermal fibroblasts and melanoma, the cells were seeded in a 6-well plate with a glass coverslip (Fisher Brand) attached to the bottom. After an incubation period of 24 hours at 37° C. in a humidified incubator with 5% carbon dioxide (CO2), media was removed and replaced with a fresh one containing a concentration of 50 μg/mL of the different synergies. Cells were cultured for another 24 hours at same conditions. After the experiments, the coverslips were fixed with a primary fixative solution containing 2.5% glutaraldehyde and 0.1 M sodium cacodylate buffer solution for 1 hour. Subsequently, the fixative solution was exchanged for 0.1 M sodium cacodylate buffer, and the coverslips were washed 3 times for 10 minutes. Post-fixation was done using 1% osmium tetroxide (OsO4) solution in buffer for 1 hour. Afterwards, the coverslips were washed three times with buffer, and dehydration was progressively achieved with 35, 50, 70, 80, 95, and 100% ethanol—three times for the 100% ethanol. Finally, the coverslips were dried by liquid CO2-ethanol exchange in a Samdri®-PVT-3D Critical Point dryer. The coverslips were mounted on SEM stubs with carbon adhesive tabs (Electron Microscopy Sciences, EMS) after treatment with liquid graphite, and then sputter coated with a thin layer of platinum using a Cressington 208HR High Resolution Sputter Coater. Digital images of the treated and untreated bacteria were acquired using SEM.

### Instruments and Characterization

A thorough morphological characterization of the synergetic structures was accomplished using transmission electron microscopy (JEM-1010 TEM, JEOL USA Inc., MA). A TEM image of the PdNPs-TENWs is shown in FIG. 1A. A TEM image of the PtNPs-TENWs is shown in FIG. 1B. To prepare samples for imaging, the nanoparticles were dried on 300-mesh, copper-coated carbon grids (Electron Microscopy Sciences, Hatfield, Pa.). An FEI Verios 460 Field Emission Microscope (FE-SEM, FEI Europe B.V., Eindhoven, Netherlands) using selective secondary/backscattered electrons detection was also used for morphological characterization. SEM images of PdNPs-TENWs and PtNPs-TENWs are shown in FIGS. 2A and 2B, respectively. Subsequent observation was done using 7 μL of a solution of PtNPs-TeNWs and PdTeNWs on distilled water, that were deposited on clean Si substrates and were allowed to dry for more than 24 hours. The images were taken with 2 kV acceleration voltage and a 25 pA electron beam current. Electron dispersive X-Ray spectroscopy (EDX) was performed using an EDX detector (EDAX Octane Plus, Ametek B.V., Tilburg, Netherlands) coupled to the SEM previously mentioned, for the verification of the presence of elemental tellurium in the structures. SEM conditions for EDX measurements were 10 kV acceleration voltage and 400 pA beam current. EDX analyses for PtNPs-TENWs are illustrated in FIGS. 3A-3B and 4A-4D. EDX analyses for PdNPs-TENWs are illustrated in FIGS. 5A-5B and 6A-6D.

Further analysis of the nanostructures was carried out by infrared spectroscopy using a Fourier transform infrared spectrometer, Perkin Elmer 400 FT-IR/FT-NIR, in attenuated total reflectance (ATR) mode. The samples for FT-IR analysis were prepared by drop casting the nanostructure colloids on a sample holder heated at ˜50° C. The IR spectra were measured in the range of 500 to 4000 cm⁻¹ with a resolution of 4 cm⁻¹. The spectra were normalized and the baseline corrected using Spectrum™ software from Perkin-Elmer. FIG. 7 shows the overlaid IR spectra for PtNPs-TENWs, PdNPs-TENWs, and potato starch.

Crystal structure characterization of the synthesized TeNWs-based nanomaterials was carried out by powder X-ray diffraction (XRD, FIG. 8). The XRD patterns were obtained with a Rigaku MiniFlex 600 operating with a voltage of 40 kV, current of 15 mA, and Cu-Kα radiation (λ=1.542 Å). All XRD patterns were recorded at room temperature with a step width of 0.05 (2θ) and scan speed of 0.2°/min. Samples for the XRD analyses were prepared by drop-casting 8 mL of the colloids onto the sample holder.

In measurement of the X-Ray photoelectron spectroscopy (XPS), drops of both compounds dispersed in water were deposited on clean copper substrates for sample preparation. After water evaporation the samples were loaded in a vacuum loadlock chamber and then transferred in the XPS Ultra High Vacuum chamber with base pressure of 10⁻¹⁰ mbar. The XPS chamber was equipped with a hemispherical electron energy Analyzer (SPECS Phoibos 100 spectrometer) and an AlKα (1486.29 eV) X-ray source. The angle between the hemispherical analyzer and the plane of the surface was kept at 60° C. Wide scan spectra were recorded using an energy step of 0.5 eV and a pass-energy of 40 eV, while specific core level spectra (Te 3d, Pd 3d, Pt 4f, O 1s and C 1s) were recorded using an energy step of 0.1 eV and a pass-energy of 20 eV. Data processing was performed with CasaXPS software (Casa software Ltd, Cheshire, UK). The absolute binding energies of the photoelectron spectra were determined by referencing to the Pt 4f_{7/2} at 71.2 eV in one of the samples and to Pd 3d_{5/2} at 335.1 eV in the other. The contributions of the AlKα satellite lines were subtracted and the spectra were normalized to the maximum intensity.

A SpectraMax M3 spectrophotometer (Molecular Devices, Sunnyvale, Calif.) was used to measure the optical density (OD) of the bacterial cultures. Growth curves and other bacterial analyses were performed in a plate reader SpectraMax® Paradigm® Multi-Mode Detection Platform.

For cell fixation studies, a Cressington 208HR High Resolution Sputter Coater and a Samdri®-PVT-3D Critical Point dryer was used to prepare the samples, that were imaged using a Hitachi S-4800 SEM instrument with a 3 kV accelerating voltage and 10 μA of current.

### Statistical Analysis

All experiments were repeated in triplicate (N=3) to ensure reliability of results. Statistical significance was assessed using student's t-tests, with a p<0.05 being statistically significant. Results are displayed as mean±standard deviation.

What is claimed is:

1. A method of inhibiting proliferation of pathogenic cells in a subject, the method comprising administering to the subject coated metal nanoparticles attached to tellurium nanowires, wherein the tellurium nanowires have a wire core comprising tellurium, and wherein the coated metallic nanoparticles have a metallic core and an outer coating comprising a polymer, whereby proliferation of the pathogenic cells is inhibited.

2. The method of claim 1, wherein the pathogenic cells are cancer cells and proliferation of the cancer cells is inhibited at least twice as much as proliferation of non-cancerous cells is inhibited in the subject.

3. The method of claim 1, wherein the pathogenic cells are bacterial cells or drug resistant bacterial cells.

4. The method of claim 1, wherein the polymer is a biopolymer isolated from a naturally occurring biological material.

5. The method of claim 4, wherein the biopolymer is starch.

6. The method of claim 1, wherein the polymer is a synthetic polymer.

7. The method of claim 6, wherein the synthetic polymer is polyethylene glycol.

8. The method of claim 1, wherein the wire core comprises tellurium hexagonal crystal structure.

9. The method of claim 1, wherein at least a portion of the tellurium nanowires have a star-shaped structure comprising tellurium nanowires radiating outward from a central point.

10. The method of claim 1, wherein the tellurium nanowires have a diameter of about 15 nm to about 35 nm.

11. The method of claim 1, wherein the coated metal nanoparticles each have a size in the range from about 35 nm to about 120 nm.

12. The method of claim 1, wherein the coated metallic nanoparticles have a coating that is about 1 nm thick.

13. The method of claim 1, wherein the metallic core comprises a metal oxide.

14. A method of producing polymer-coated metal nanoparticles attached to tellurium nanowires, the method comprising:
(a) mixing telluric acid (H₂TeO₄) with an aqueous polymer solution or suspension to obtain a mixture of telluric acid, polymer, and water;
(b) heating the mixture in a sealed vessel at a temperature from about 120° C. to about 200° C. for about 2 hours to about 20 hours; whereby tellurium nanowires coated with the polymer are produced;
(c) centrifuging the product from step (b) to obtain a pellet;
(d) resuspending the pellet in water; and
(e) adding a metallic salt to the suspension and waiting for a reaction time, whereby polymer-coated metal nanoparticles attached to tellurium nanowires are produced.

15. The method of claim 14, further comprising:
(f) centrifuging the product from step (e) to obtain a pellet;
(g) resuspending the pellet in water; and
(h) lyophilizing the resuspended pellet.

16. The method of claim 14, wherein the reaction time is about 1 minute.

17. The method of claim 14, wherein the polymer-coated metal nanoparticles comprise a metal oxide.

18. The method of claim 14, wherein the metallic salt is palladium chloride (PdCl₂), potassium tetrachloroplatinate (K₂PtCl₄), or combinations thereof.

19. The method of claim 14, wherein the polymer is starch.

20. The method of claim 14, wherein the temperature in step (b) is about 160° C.

21. The method of claim 14, wherein the heating in step (b) is carried out for about 15 hours.

22. The method of claim 14, wherein the tellurium nanowires coated with the polymer are produced in the form of star-shaped nanostructures, each star-shaped nanostructure comprising a central cluster and a plurality of coated tellurium nanowires extending from the central cluster.

23. The method of claim 14, wherein the resulting tellurium nanowires each have a diameter of about 15 nm to about 35 nm.

24. The method of claim 14, wherein the coated metal nanoparticles each have a size in the range from about 35 nm to about 120 nm.

25. The method of claim 14, further comprising;
(e1) sonicating the polymer-coated metal nanoparticles attached to tellurium nanowires to release the polymer-coated metal nanoparticles from the tellurium nanowires.

26. The method of claim 19, wherein the produced tellurium nanowires comprise a core comprising tellurium in a hexagonal tellurium crystal form and the metallic nanoparticles have a coating comprising starch.

27. Tellurium nanowires having a core comprising tellurium and metallic nanoparticles with an outer coating comprising a polymer made by a method of any one of claims 14-24.

28. The tellurium nanowires of claim 27, wherein the tellurium nanowires do not comprise amorphous tellurium.
